(19) United States
(12) Patent Application Publication
(10) Pub. No.: US 2026/0351350 A1
(43) Pub. Date: Nov. 5, 2026

(54) QUANTITATIVE AFFINITY ACTIVITY SPECIFIC CELL ENRICHMENT

(71) Applicant: ABSCI CORPORATION, Vancouver, WA (US)

(72) Inventors: Miles Gander, Vancouver, WA (US); Roberto Spreafico, Vancouver, WA (US); John Sutton, Vancouver, WA (US); Matthew Weinstock, Camas, WA (US); David Spencer, Vancouver, WA (US)

(21) Appl. No.: 19/103,738

(22) PCT Filed: Aug. 14, 2023
(86) PCT No.: PCT/US2023/072153

§ 371 Date: Feb. 13, 2025

Related U.S. Application Data
(60) Provisional application No. 63/371,474, filed on Aug. 15, 2022.

(51) Int. Cl. G16B 40/20; G06N 20/00; G16B 20/50; G16B 35/10; G16B 35/20

(52) U.S. Cl. CPC G16B 40/20; G16B 20/50; G16B 35/10; G16B 35/20; G06N 20/00

(57) ABSTRACT

The present disclosure generally relates to methods of rapidly and efficiently searching biologically-related data space. More specifically, the disclosure includes methods of identifying biomolecules with desired properties, or which are most suitable for acquiring such properties, from complex biomolecule libraries or sets of such libraries. The disclosure also provides methods of modeling sequence-activity relationships. The disclosure further relates to methods for generating training data for training a machine learning model to predict biomolecule gene sequences with desired properties. Methods of the present disclosure have utility in the optimization of proteins for industrial and therapeutic use.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “57890_Seqlisting.XML”, which was created on Aug. 3, 2023, and is 7,223 bytes in size. The subject matter of the Sequence Listing is incorporated herein in its entirety by reference.

### FIELD

The present disclosure relates to the fields of molecular biology, molecular evolution, bioinformatics, and digital systems. More specifically, the disclosure relates to methods for generating training data for training a machine learning model to predict biomolecule sequences with desired properties. Methods of the present disclosure have utility in the optimization of proteins for industrial and therapeutic use.

### BACKGROUND

Biomolecule engineering aims to identify biomolecule variants with new, surprising or enhanced functional properties. A common approach for biomolecule engineering, directed biomolecule evolution, involves in vitro diversity generation where high rates of mutations are imposed on a parent biomolecule sequence, while applying selective pressure. This is typically achieved by the generation of libraries of biomolecule variants (for example by error-prone PCR), expression of the libraries in cells, screening of these libraries, and selection of biomolecule variants with desired properties or functions. Improved variants selected from such a screening serve as templates for the next round of in vitro diversification, expression and selection or screening, advancing the evolutionary cycle and through subsequent repetition of the process using improved variants from the previous screening.

A major challenge for the success of this approach remains that the total number of variant sequences and mutation combinations that could be generated is many orders of magnitude larger than what it is possible to express and screen in the laboratory. This concept is described as sequence space (Currin et al. Chem. Soc. Rev. 44:1172-1239 (2015); Hayashi et al. PLOS One. 1 (1): e96 (2006); Povolotskaya and Kondrashov. Nature. 465:922-926 (2010); Wong et al. Biocatal Biotransformation. 25:229-241 (2007)), which describes the total number of possible variants of a given sequence of length, n. Sequence space is vast and experimentally untestable for even very small protein sequences, for example the sequence space of a small 100 amino acid protein is ~1.3×10¹³⁰ (20¹⁰⁰, given the 20 possible amino acids) (Kondrashov and Kondrashov. Trends Genet. 31:24-33 (2015)). Since the search space grows exponentially with the number of amino acid positions considered (combinatorial explosion), functional variants are extremely rare in this vast space of variant sequences, and experimental screening can be resource-intensive, with researchers often limited to testing a few hundred or thousand variants. This limits functional improvements that are obtainable in a reasonable time.

To reduce experimental effort associated with directed protein evolution while expanding the reach and better explore the sequence space encoded by mutating multiple positions simultaneously, machine learning (ML) can be incorporated into the directed evolution workflow. In this method, a ML model is trained to learn the sequence-function relationship from sequence and screening data. In this method, all variants in a diversity library generated by saturation mutagenesis and/or random mutagenesis are experimentally evaluated to obtain their sequences and functions. The sequence and screening data obtained, including from those of unimproved variants, are then used as training data to construct a ML model that predicts the function from the sequence. By using the ML model, a second-round library that contains variants predicted to have improved functions is generated. This method enables the design of a library with high enrichment of desirable variants and thus has been successfully applied to directed evolution of various proteins including fluorescent proteins, (Saito et al. ACS Synth. Biol. 7:2014-2022 (2018); Alley et al. Nat. Methods. 16:1315-1322 (2019); Biswas et al. Nat. Methods. 18:389-396 (2021)), enzymes, (Liao et al. BMC Biotechnol. 27:16 (2017); Fox et al., Nat. Biotechnol. 25:338-344 (2007); Wu et al. Proc. Natl. Acad. Sci. U.S.A 2116:8852-8858 (2019)), and others (Giguère et al. PLOS Comput. Biol. 11: e10040742015 (2015); Bedbrook et al. Nat. Methods. 16:1176-1184 (2019)).

Affinity maturation of antibodies is an important capability for drug discovery. Maturation is commonly done by mutagenizing an existing binding candidate and screening for increased binding. A challenge with mutagenic wet lab techniques is that mutagenic space is quite vast and screening of individual clones with methods such as SPR and BLI are low throughput, restricting the observable sequence space. Additionally, most mutants based on a starting antibody will reduce or abolish binding, making observation of a range of affinities potentially difficult. On the other hand, technologies such as phage display allow selection of binders in a large sequence space but are unable to provide quantitative readouts regarding affinity of individual mutants which is vital for generating data to support AI model generation.

### SUMMARY

In some embodiments, the present disclosure provides methods for generating training data. In one embodiment, a method for generating training data for a machine learning model is provided comprising:
a) expressing a biomolecule variant library in host cells;
b) measuring: (i) expression levels and (ii) affinity values to a binding partner of interest of two or more biomolecule variants expressed in (b);
c) sorting the host cells into a distribution of cell subpopulations based on the measured expression levels and measured affinity values; thereby collecting cells across an affinity distribution;
d) sequencing the biomolecule variants expressed from the collected cells of (c);
e) calculating an enrichment score for each sequenced biomolecule variant, wherein said enrichment score and said biomolecule variant sequence is capable of training a machine learning model capable of performing sequence-based affinity predictions.

In another embodiment, an aforementioned method is provided wherein the library of biomolecule variants is generated by randomly mutating a nucleic acid encoding a reference biomolecule. In another embodiment, an aforementioned method is provided wherein the library of biomolecule variants is generated by random mutagenesis, error-prone PCR mutagenesis, oligonucleotide-directed mutagenesis, cassette mutagenesis, shuffling, saturation mutagenesis, homology-directed mutagenesis, Activation Induced Cytidine Deaminase (AID) mediated mutagenesis, or transposon mutagenesis. In still another embodiment, an aforementioned method is provided wherein the library of biomolecule variants comprises at least 10⁴-10⁷ unique biomolecule variant sequences. In yet another embodiment, an aforementioned method is provided wherein the library of biomolecule variants are displayed on the host cell surface. In another embodiment, an aforementioned method is provided wherein the library of biomolecule variants are expressed and retained in the host cell cytoplasm.

In another embodiment, an aforementioned method is provided wherein the host cells are Escherichia coli cells. In yet another embodiment, an aforementioned method is provided wherein Escherichia coli cells are Escherichia coli 521 cells. In another embodiment, an aforementioned method is provided wherein the Escherichia coli cells comprises one or more or all of:
a) an alteration of gene function of at least one gene encoding a transporter protein for an inducer of at least one inducible promoter;
b) a reduced level of gene function of at least one gene encoding a protein that metabolizes an inducer of at least one inducible promoter;
c) a reduced level of gene function of at least one gene encoding a protein involved in biosynthesis of an inducer of at least one inducible promoter;
d) an altered gene function of a gene that affects the reduction/oxidation environment of the host cell cytoplasm;
e) a reduced level of gene function of a gene that encodes a reductase;
f) at least one expression construct encoding at least one disulfide bond isomerase protein;
g) at least one polynucleotide encoding a form of DsbC lacking a signal peptide; and/or
h) at least one polynucleotide encoding Ervlp.

In still another embodiment, an aforementioned method is provided wherein step (c) optionally additionally measures one or more of binding specificity, biological activity, stability, and/or solubility of the expressed biomolecule variants.

In yet another embodiment, an aforementioned method is provided wherein affinity is quantified by measuring binding dissociation constant (K_{D}) of a biomolecule variant to the binding partner of interest. In one embodiment, the binding partner of interest is a fluorescently labeled antigen.

In still another embodiment, an aforementioned is provided wherein expression level of the biomolecule variants is quantified by measuring anti-IgG-binding capacity. In another embodiment, an aforementioned is provided wherein expression level of the biomolecule variants is quantified using an anti-IgG antibody conjugated to a fluorophore. In yet another embodiment, an aforementioned is provided wherein expression level of the biomolecule variants is quantified by measuring a non-antigen binding capacity.

The present disclosure also provides, in some embodiments, an aforementioned method wherein the measuring in step (c) and sorting in step (d) comprises a fluorescence-activated cell sorting (FACS) assay. In another embodiment, an aforementioned is provided optionally further comprising measuring binding affinity of the sequenced biomolecule variants prior to calculating an enrichment score. In one embodiment, the binding affinity is measured using an assay selected from the group consisting of a Surface Plasmon Resonance (SPR) based binding assay, Biolayer Interferometry and/or flow cytometry derived binding curves.

In another embodiment, an aforementioned is provided wherein the sequencing of step (e) is obtained by a method selected from the group consisting of deep sequencing, next generation sequencing, Long read nanopore sequencing, Single Molecule Real-Time long read sequencing (pacbio). In another embodiment, the sequencing of step (e) is obtained by a method selected from the group consisting of deep sequencing, next generation sequencing, Long read nanopore sequencing, Single Molecule Real-Time long read sequencing (pacbio). In another embodiment, an aforementioned is provided wherein wherein nucleic acids encoding the biomolecule variants are modified prior to sequencing to comprise barcode sequences comprising unique molecular identifiers (UMIs).

The present disclosure also provides, in one embodiment, an aforementioned method wherein the biomolecule variants are selected from a group consisting of a monoclonal antibody, a bispecific antibody, a multispecific antibody, a humanized antibody, a chimeric antibody, a camelid antibody, a single domain antibody, a single-chain Fvs (ScFv), a single chain antibody, a Fab fragment, a F(ab′) fragment, a disulfide-linked Fvs (sdFv), or an anti-idiotypic (anti-Id) antibody. In another embodiment, an aforementioned is provided wherein wherein the biomolecule variants are selected from a group consisting of a monoclonal antibody, a bispecific antibody, a multispecific antibody, a humanized antibody, a chimeric antibody, a camelid antibody, a single domain antibody, a single-chain Fvs (ScFv), a single chain antibody, a Fab fragment, a F(ab′) fragment, a disulfide-linked Fvs (sdFv), or an anti-idiotypic (anti-Id) antibody. In yet another embodiment, an aforementioned is provided wherein the biomolecule variants are selected from a group consisting of a peptide, a polypeptide, a protease, an oxidoreductase, a transferase, a hydrolase, a lyase, an isomerase, a ligase, an enzyme, an antibody, a cytokine, a chemokine, a nucleic acid, a metabolite, a small molecule (<1 kDa) and a synthetic molecule.

In still another embodiment, a method for generating training data for a machine learning model is provided comprising:
a) expressing a biomolecule variant library in host cells;
b) measuring: (i) expression levels and (ii) affinity values to a binding partner of interest of two or more biomolecule variants expressed in (b);
c) sorting the host cells into a distribution of cell subpopulations based on the measured expression levels and measured affinity values; thereby collecting cells across an affinity distribution;
d) isolating nucleic acids encoding the biomolecule variants from the collected host cells of (c), amplifying said nucleic acids using selective rolling circle amplification (sRCA), and sequencing nucleic acids encoding the biomolecule variants; and
e) calculating an enrichment score for each sequenced biomolecule variant, wherein said enrichment score and said biomolecule variant sequence is capable of training a machine learning model capable of performing sequence-based affinity predictions.

### BRIEF DESCRIPTION OF THE DRAWINGS

FIG. 1 is a schematic showing a ML guided affinity maturation workflow that uses qaACE data and Carterra SPR data for generation of an affinity prediction model for drug variants, according to one embodiment. FIG. 1A-qaACE for data-centric AI FIG. 1B-A schematic representation of the qaACE assay workflow. Libraries of antibody variants are expressed in SoluPro™ E. coli B Strain. Cells are fixed, permeabilized and stained with fluorescently labeled antigen and scaffold probes. Cells are then sorted based on expression and affinity levels, followed by sequencing and qaACE affinity score computation.

FIG. 2 shows qaACE correlation with SPR affinity measurements as well as prediction performance of deep language models for antibody binding affinity trained on qaACE data. (FIG. 2A) Correlation of qaACE scores vs. SPR measured affinity. FAB variants from trast-1 (see Table 1) single and double mutant library were used to generate qaACE score and shared variants from trast-2 were used to generate the correlation. (FIG. 2B) Correlation of ML model predictions of qaACE scores trained on qaACE data from single and double mutants in trast-1 library. (FIG. 2C) Correlation of ML model predictions of qaACE scores trained on qaACE data from up to triple mutants in trast-3 library.

FIG. 3 shows the specificity of the sRCA amplification of a plasmid backbone expressing one of three antibiotic resistance markers: PL2945 (Kan), PL3133 (Chlor), and PL3137 (Carb). Reactions were conducted in triplicate. Each amplification was subsequently sequenced, aligned to the components of the plasmid, and the percentage of reads mapping to each element was calculated. The percentage of reads for each plasmid type are graphed in FIG. 3A, including the off-target genomic reads. The raw data for the reactions conducted in triplicate are presented in FIG. 3B.

FIG. 4 shows the results of an alternative sample preparation method to the standard miniprep for reducing off-target genomic reads by using PlasmidSafe and PippenHT size selection. As shown in the graph in FIG. 4A and the raw data in FIG. 4B, both PlasmidSafe treatment and PippenHT size selection greatly improve the specificity of the reaction over the base miniprep or sRCA alone. However, the best results were obtained from the sRCA treatment combined with PippenHT size selection.

FIG. 5 shows the ability of Phi29 DNA polymerase to conduct the amplification reaction in the presence of increasing concentrations of PBS. Sheath fluid from the FACSymphony is PBS, and in higher concentrations the salts are inhibitory to the amplification reaction. Pre-processing the sorted samples to dilute and remove as much salt as possible allowed for a greater percentage of plasmid amplification by Phi29 DNA polymerase.

FIG. 6 shows a flow cytometry gating scheme. After parent gating to reduce aggregates, debris, and non-permeabilized cells, bias to antigen binding signal from expression variability was controlled through an additional parent gate on the 30% mid expressers. Six collection gates were then used to bin evenly across the log range of the antigen signal (sort option 1). Alternatively, cells may be collected on the ratio of the expression signal over binding signal (sort option 2).

FIG. 7 shows that qaACE scores are highly correlated with SPR measured KDS across multiple leads and antigens.

### DETAILED DESCRIPTION

A major challenge for constructing accurate machine-learning models is the scarcity of appropriate large-scale training datasets. Directed evolution platforms are well-suited for this as they rely on the linking of biological sequence data (DNA, RNA, protein) to a phenotypic output. In fact, it has long been proposed to use ML models trained on data generated by mutagenesis libraries as a means to guide protein engineering. In recent years, access to deep sequencing and parallel computing has enabled the construction of deep learning models capable of predicting molecular phenotype from sequence data. Deep learning incorporates multiple hidden layers to decipher relationships buried in large, high-dimensional data sets, such as the millions of reads gathered from a single deep sequencing experiment. Well trained models can then be used to make predictions on completely unseen and novel variants. This application of model extrapolation lends itself perfectly to protein engineering because it provides a way to interrogate a much larger sequence space than what is physically possible. Here we address this problem by combining deep mutational scanning and a bacterial display system to generate a training dataset for A ML model to learn sequence-function relationships.

An activity-specific cell-enrichment (ACE) assay that identifies host cells that express active gene product of interest (e.g., biomolecules, as used herein) rather than inactive material, has been described in WO 2021/146626, incorporated herein in relevant part. Active gene products can be distinguished from inactive material by the ability of active gene products to specifically bind a binding partner molecule, or by the ability of gene products to participate in a chemical or enzymatic reaction, as examples. The presence of properly formed disulfide bonds in a polypeptide gene product is an indication that it is correctly folded and presumptively active. In the cell-enrichment methods, active gene product of interest is detected by utilizing an appropriate labeling complex that specifically binds to active gene product of interest, such as a labeled antigen if the gene product of interest is an antibody or Fab; or a labeled ligand if the gene product of interest is a receptor or a receptor fragment, where the ligand specifically binds to an active conformation of the receptor; or a labeled substrate or a labeled substrate analog if the gene product of interest is an enzyme, as examples. For any gene product of interest, if there is an available antibody or antibody fragment that specifically binds to the active gene product and not to inactive gene product, that antibody or antibody fragment can be used to label the active gene product of interest when attached to a detectable moiety.

A key strength of ACE is its ability to screen tens of thousands of “units of variation” in a single run. However, ongoing AI efforts applied to drug discovery add additional requirements to wet lab-only screenings, which impose additional optimization of ACE to generate datasets suitable for AI Wet lab-only screenings aimed at selecting top performing variants do not require stringent quantitativeness from an assay. Indeed, the iterative nature of such screenings is such that hits from the n−1 step are rescreened in step n, effectively weeding out n−1 false positives. Moreover, wet lab screenings are often tuned to selecting only a desired population of interest (for example, higher affinity variants), and as such the assay does not have to be quantitative over a large dynamic range of the parameter of interest (for example, antibody affinity). However, AI models for predicting quantitative predictions benefit from quantitative sequence variant training data. As such, quantitative sequence variant training data need to be accurate for the model to produce meaningful predictions down the line. The present disclosure addresses these needs and shortcomings.

The present disclosure provides, in various embodiments, an augmentation of the ACE assay-quantitative affinity ACE (“qaACE”), as a method for sampling the affinity of antibody variants at high throughput using flow cytometry and next generation sequencing to generate a qaACE score that correlates with K_{D}. The main goal of this method is to generate highly quantitative high throughput training data for an AI model to perform sequence-based affinity predictions. This method can be applied to any antibody format, mabs, fabs, scFv, scFAB, VHH, nanobody etc. and could conceivably be applied to other binding drug formats as well.

In one embodiment, the first step in the qaACE process is to generate a mutationally diverse antibody library that evenly samples the sequence space around the starting point antibody molecule. This library contains variants that span a range in mutational distance from the original sequence.

In some embodiments including the Examples herein, the method provides a flow cytometry read out of an antibody, expressed in SoluPro E. coli, binding to a fluorescently labeled antigen probe. In the qaACE assay, expression of the antibody molecule is normalized so that a change in fluorescent signal in a cell will be due to different affinities of the expressed antibody variants in the cells binding to the fluorescent antigen probe. This normalization is accomplished via a generic target molecule probe that will bind to all variants and whose signal will be in an orthogonal fluorescent channel to the antigen probe. In this setting we show that the fluorescent signal of a variant is proportional to the measured K_{D} of an antibody variant. Given this proportionality, using FACS, cells containing antibody variants can be sorted that span a range (e.g., a distribution) of affinities.

After binning cells across the fluorescence range representing antigen binding, the associated DNA is isolated and sequenced. Sequencing reads for each library variant are quantified and the ACE score calculated as the normalized average number of reads across collection gates. The ACE scores generated via qaACE are an ideal data type for AI modeling purposes because they represent a high throughput proxy for affinity equilibrium constants (KDs).

In one exemplary workflow, the present disclosure provides a qaACE assay that comprises some or all of the following general steps:
1) Generation of an antibody or other drug molecule library for screening through qaACE expressed in a host cell such as SoluPro E. coli.
2) Identification of an antigen or binding partner probe that is fluorescently labeled for use in, for example, FACS via the initial cytometry development process.
3) Use of generic probe to the target molecule variants that will allow for detection of expression level within a cell. This expression signal is used to gate a uniformly expression population to disambiguate affinity and expression signal related to epitope binding signal.
4) Sorting of cells across the affinity distribution.
5) Sequencing of cells sorted across the affinity distribution.
6) During the sequencing DNA barcodes or UMIs may be added via PCR amplification of the region of interest. These UMIs will enable absolute quantification of variants retrieved from the gates.
7) Generation of affinity correlated enrichment scores for each observed variant.
8) AI model training using ACE score and antibody variant sequence.

As described herein, the present disclosure provides a method for generating highly quantitative high-throughput training data for a ML model to perform, for example, sequence-based affinity predictions. In some embodiments of the present disclosure, sequences of a highly diverse library of biomolecule variants, which are expressed in, or on the surface of, host cells, serve as input to an experiment (e.g., an assay to determine expression and/or affinity, among other readouts). In some embodiments, the variants are sorted into a plurality of bins based on high throughput measurements of binding affinity values (K_{D}) which are normalized for variant expression levels and variant sequences in each bin are obtained and tallied by deep DNA sequencing. In some embodiments, the method then outputs a plurality of enrichment scores which correlate the K_{D} across the full experimental affinity distribution, (i.e. from non-binders, low and high binders) and sequence information of every biomolecule variant in each bin. The enrichment scores generated via qaACE assay of the present disclosure are an ideal data type for AI modeling purposes because of their accuracy and throughput. The combined method of obtaining affinity and sequence data of biomolecule variants is accordingly referred to herein as the quantitative affinity Activity-specific Cell Enrichment (qaACE) assay.

As used herein, the term “quantitative affinity Activity-specific Cell Enrichment or qaACE assay” refers to a high throughput assay for obtaining affinity and sequence data of biomolecule variants.

As used herein, the term “affinity distribution” refers to the distribution of K_{D}values for antigen binding to all possible sequence variants in the randomized library of biomolecule variants. A comparison to the K_{D}value of the reference biomolecule gives an indication whether the variants bind with a higher or lower affinity.

In some embodiments, the present disclosure provides a method for generating training data for a machine learning model comprising the steps of:
a. expressing a biomolecule variant library in host cells;
b. measuring: (i) expression levels and (ii) affinity values to a binding partner of interest of two or more biomolecule variants expressed in (b);
c. sorting the host cells into a distribution of cell subpopulations based on the measured expression levels and measured affinity values; thereby collecting cells across an affinity distribution;
d. sequencing the biomolecule variants expressed from the collected cells of (c); and
e. calculating an enrichment score for each sequenced biomolecule variant, wherein said enrichment score and said biomolecule variant sequence is capable of training a machine learning model capable of performing sequence-based affinity predictions.

In some embodiments, the present disclosure provides a method for sampling affinities of a plurality of biomolecule variants (e.g., for training data for a machine learning model) comprising the steps of:
a. expressing a biomolecule variant library in host cells;
b. measuring: (i) expression levels and (ii) affinity values to a binding partner of interest of two or more biomolecule variants expressed in (b);
c. sorting the host cells into a distribution of cell subpopulations based on the measured expression levels and measured affinity values; thereby collecting cells across an affinity distribution;
d. sequencing the biomolecule variants expressed from the collected cells of (c); and
e. calculating an enrichment score for each sequenced biomolecule variant, wherein said enrichment score and said biomolecule variant sequence is capable of training a machine learning model capable of performing sequence-based affinity predictions.

In some embodiments, the present disclosure provides a method for sorting cells that express a plurality of biomolecule variants capable of binding a target antigen across a range of affinities (e.g., to generate training data for a machine learning model) comprising the steps of:
a. expressing a biomolecule variant library in host cells;
b. measuring: (i) expression levels and (ii) affinity values to a binding partner of interest of two or more biomolecule variants expressed in (b);
c. sorting the host cells into a distribution of cell subpopulations based on the measured expression levels and measured affinity values; thereby collecting cells across an affinity distribution;
d. sequencing the biomolecule variants expressed from the collected cells of (c); and
e. calculating an enrichment score for each sequenced biomolecule variant, wherein said enrichment score and said biomolecule variant sequence is capable of training a machine learning model capable of performing sequence-based affinity predictions.

In some embodiments, the present disclosure provides a method for generating an affinity distribution for a plurality of biomolecule variants (e.g., to generate training data for a machine learning model) comprising the steps of:
a. expressing a biomolecule variant library in host cells;
b. measuring: (i) expression levels and (ii) affinity values to a binding partner of interest of two or more biomolecule variants expressed in (b);
c. sorting the host cells into a distribution of cell subpopulations based on the measured expression levels and measured affinity values; thereby collecting cells across an affinity distribution;
d. sequencing the biomolecule variants expressed from the collected cells of (c); and
e. calculating an enrichment score for each sequenced biomolecule variant, wherein said enrichment score and said biomolecule variant sequence is capable of training a machine learning model capable of performing sequence-based affinity predictions.

### Biomolecules

In some embodiments, the first step of the methods of the current disclosure comprise the expression of a biomolecule variant library in host cells.

As used herein, the term “biomolecule” or “biological molecule” refers to a molecule that is generally found in a biological organism. Typical biomolecules include, but are not limited to, RNA, DNA, peptides, polypeptides or proteins, lipids, carbohydrates, or other organic molecules. The term “biomolecule variants” as used herein, refers to new biomolecules whose sequences differ from the sequence of a parental biomolecule through mutations that are introduced according to the methods of the disclosure.

As used herein, the term “parental polypeptide,” “parental polynucleotide,” “parent nucleic acid,” and “parent” are generally used to refer to the wild-type polypeptide, wild-type polynucleotide, or a variant used as a starting point in a diversity generation procedure such as a directed evolution. In some embodiments, the parent itself is produced via shuffling or other diversity generation procedure. In some embodiments, mutants used in directed evolution are directly related to a parent polypeptide. In some embodiments, the parent polypeptide is stable when exposed to extremes of temperature, pH and/or solvent conditions and can serve as the basis for generating variants for shuffling. In some embodiments, the parental polypeptide is not stable to extremes of temperature, pH and/or solvent conditions, and the parental polypeptide is evolved to make robust variants.

As used herein, the term “directed evolution” or “artificial evolution” refers to the modification and improvement of biomolecule function by mimicking “Darwinian selection” through iterations of mutation and screening or selection for improved properties. After each step, the most promising candidates are used as templates for a new round of mutation and screening or selection. This strategy can be repeated until the desired features are obtained.

In some embodiments, the biomolecule is an antibody, an enzyme, a hormone, a cytokine, growth factor, clotting factor, anticoagulation factor, albumin, antigen, an adjuvant, a transcription factor, or a cellular receptor.

Cytokines include, but are not limited to, chemokines, interferons, interleukins, lymphokines, and tumor necrosis factors. Cellular receptors, such as cytokine receptors, also are contemplated. Examples of cytokines and cellular receptors include, but are not limited to, tumor necrosis factor alpha and beta and their receptors; lipoproteins; colchicine; corticotropin; vasopressin; somatostatin; lypressin; pancreozymin; leuprolide; alpha-1-antitrypsin; atrial natriuretic factor; thrombin; enkephalinase; RANTES (regulated on activation normally T-cell expressed and secreted); human macrophage inflammatory protein (MIP-1-alpha); cell determinant proteins such as CD-3, CD-4, CD-8, and CD-19; erythropoietin; interferon-alpha, -beta, -gamma, -lambda; colony stimulating factors (CSFs), e.g., M-CSF, GM-CSF, and G-CSF; IL-1, 2, 3, 4, 5, 6, 7, 8, 9 and/or IL-10; T-cell receptors; and prostaglandin.

Examples of hormones include, but are not limited to, antidiuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone, aldosterone, cortisol, epinephrine, glucagon, insulin, estrogen, progesterone, and testosterone.

Examples of growth factors include, e.g., vascular endothelial growth factor (VEGF), nerve growth factor (NGF), platelet-derived growth factor (PDGF), fibroblast growth factor (FGF), epidermal growth factor (EGF), transforming growth factor (TGF), bone morphogenic proteins (BMPs), and insulin-like growth factor-I and -II (IGF-I and IGF-II).

Examples of clotting factors or a coagulation factors include Factor I, Factor II, Factor III, Factor V, Factor VI, Factor VII, Factor VIII, Factor VIIIC, Factor IX, Factor X, Factor XI, Factor XII, Factor XIII, von Willebrand factor, prekallikrein, heparin cofactor II, antithrombin III, and fibronectin.

Examples of enzymes include, but are not limited to, angiotensin converting enzyme, streptokinase, L-asparaginase, and the like. Other examples of enzymes include, e.g., nitrate reductase (NADH), catalase, peroxidase, nitrogenase, phosphatase (e.g., acid/alkaline phosphatases), phosphodiesterase I, inorganic diphosphatase (pyrophosphatase), dehydrogenase, sulfatase, arylsulfatase, thiosulfate sulfurtransferase, L-asparaginase/L-glutaminase, beta-glucosidase, aryl acylamindase, amidase, invertase, xylanase, cellulose, urease, phytases, carbohydrase, amylase (alpha-amylase/beta-amylase), arabinoxylanase, beta-glucanase, alpha-galactosidase, beta-mannanase, pectinase, non-starch polysaccharide degrading enzymes, endoproteases, exoproteases, lipases, cellulases, oxidoreductases, ligases, synthetases (e.g., aminoacyl-transfer RNA synthetase; glycyl-tRNA synthetase), transferases, hydrolases, lyase (e.g., decarboxylases, dehydratases, deaminases, aldolases), isomerases (e.g., triose phosphate isomerase), and trypsin. Further examples of enzymes include catalases (e.g., alkali-resistant catalases), alkaline amylase, pectinase, oxidase, laccases, proxidases, xylanases, mannanases, acylases, alcalase, alkylsulfatase, cellulolytic enzymes, cellobio-hydrolase, cellobiase, exo-1,4-beta-D-glucosidase, chloroperoxidase, chitinase, cyanidase, cyanide hydratase, I-Galactono-lactone oxidase, lignin peroxidase, lysozyme, mn-peroxidase, muramidase, parathion hydrolase, pectinesterase, peroxidase, and tryosinase. Further examples of enzymes include nuclease (e.g., endonuclease, such as zinc finger nucleases, transcription activator-like effector nuclease, Cas nucleases, engineered meganucleases).

In an embodiment, the biomolecule is an antibody. The term “antibody” refers to an intact antigen-binding immunoglobulin. In various embodiments, an intact antibody comprises two full-length heavy chains and two full-length light chains. In a full-length antibody, each heavy chain consists of a heavy chain variable region (abbreviated herein VH) and a heavy chain constant region. The heavy chain constant region is comprised of three domains, CH1, CH2 and CH3. Each light chain is comprised of a light chain variable region (abbreviated herein VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

In an embodiment, the biomolecule is an “antigen-binding fragment” of an antibody. Examples of antigen-binding fragments of antibodies include, but are not limited to (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) an Fv fragment consisting of the VL and VH domains of a single arm of an antibody, (v) a dAb fragment (Ward et al., (1989) Nature 341:544-546, Winter et al., PCT Publication No. WO 90/05144), which comprises a single variable domain. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies (scFv) are also intended to be encompassed within the term “antigen-binding fragment” of an antibody.

The architecture of antibodies has been exploited to create a growing range of alternative formats that span a molecular-weight range of at least about 12-150 kDa and have a valency (n) range from monomeric, to dimeric, to trimeric, to tetrameric, and potentially higher; such alternative formats are referred to herein as “antibody-like constructs.” Antibody-like protein constructs include those based on the full antibody structure and those that mimic antibody fragments which retain full antigen-binding capacity, e.g., scFvs, Fabs, and VHH. The smallest antigen-binding fragment that retains its complete antigen binding site is the Fv fragment, which consists entirely of variable (V) regions. Other antibody-like protein constructs include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). A building block that is frequently used to create different antibody formats is the single-chain variable (V)-domain antibody fragment (scFv), which comprises V domains from the heavy and light chain (VH and VL domain) linked by a peptide linker of ~15 amino acid residues. A peptibody or peptide-Fc fusion is yet another antibody-like construct protein product. The structure of a peptibody consists of a biologically active peptide grafted onto an Fc domain. Peptibodies are described in the art. See, e.g., Shimamoto et al., mAbs 4 (5): 586-591 (2012). Other antibody-like protein constructs include a single chain antibody (SCA), a diabody, a triabody, a tetrabody, and the like.

In an embodiment, the biomolecule may be a multi-specific antibody (e.g., a bispecific antibody or trispecific antibody) having the CDR sequences set forth herein. Bispecific antibody products can be divided into five major classes: BsIgG, appended IgG, BsAb fragments (e.g., bispecific single chain antibodies), bispecific fusion proteins (e.g., antigen binding domains fused to an effector moiety), and BsAb conjugates. See, e.g., Spiess et al., Molecular Immunology 67 (2) Part A: 97-106 (2015). Examples of bispecific antibody constructs include, but are not limited to, tandem scFvs and Fab2 bispecifics. See, e.g., Chames & Baty, 2009, mAbs 1[6]: 1-9; and Holliger & Hudson, 2005, Nature Biotechnology 23[9]: 1126-1136; Wu et al., 2007, Nature Biotechnology 25[11]: 1290-1297; Michaelson et al., 2009, mAbs 1[2]: 128-141; International Patent Publication No. WO 2009032782 and WO 2006020258; Zuo et al., 2000, Protein Engineering 13[5]: 361-367; U.S. Patent Application Publication No. 20020103345; Shen et al., 2006, J Biol Chem 281[16]: 10706-10714; Lu et al., 2005, J Biol Chem 280[20]: 19665-19672; and Kontermann, 2012 MAbs 4 (2): 182, all of which are expressly incorporated herein. Multispecific antibody constructs, such as trispecific antibody constructs (including three binding domains) or constructs having more than three (e.g., four, five, or more) specificities also are contemplated.

The antibodies (or antigen-binding fragments thereof or antibody-like protein constructs) may be a human antibody (i.e., having one or more variable and constant regions derived from human immunoglobulin sequences), humanized (i.e., have a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject), or chimeric (i.e., containing one or more regions from one antibody and one or more regions from one or more other antibodies).

Suitable methods of making antibodies are known in the art. For instance, standard hybridoma methods are described in, e.g., Harlow and Lane (eds.), Antibodies: A Laboratory Manual, CSH Press (1988), and C A. Janeway et al. (eds.), Immunobiology, 5th Ed., Garland Publishing, New York, NY (2001)). Monoclonal antibodies for use in the methods of the disclosure may be prepared using any technique which provides for the production of antibody molecules by continuous cell lines in culture. These include but are not limited to the hybridoma technique originally described by Koehler and Milstein (Nature 256:495-497, 1975), the human B-cell hybridoma technique (Kosbor et al., Immunol Today 4:72, 1983; Cote et al., Proc Natl Acad Sci 80:2026-2030, 1983) and the EBV-hybridoma technique (Cole et al., Monoclonal Antibodies and Cancer Therapy, Alan R Liss Inc, New York N.Y., pp 77-96, (1985). Alternatively, other methods, such as EBV-hybridoma methods (Haskard and Archer, J. Immunol. Methods, 74 (2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1). Antibodies may also be produced by inducing in vivo production in the lymphocyte population or by screening recombinant immunoglobulin libraries or panels of highly specific binding reagents as disclosed in Orlandi et al (Proc Natl Acad Sci 86:3833-3837; 1989), and Winter G and Milstein C (Nature 349:293-299, 1991). If the full sequence of the antibody or antigen-binding fragment is known, then methods of producing recombinant proteins may be employed. See, e.g., “Protein production and purification” Nat Methods 5 (2): 135-146 (2008). In some embodiments, the antibodies (or antigen binding fragments) are isolated from cell culture or a biological sample if generated in vivo.

Exemplary antibodies or antibody targets that can be used with the methods described herein include, but are not limited to, Activase® (Alteplase); alirocumab (anti-PCSK9 monoclonal antibody designated as H1H316P, see U.S. Pat. No. 8,062,640); Aranesp® (Darbepoetin-alfa), Epogen® (Epoetin alfa, or erythropoietin); Avonex® (Interferon β-1a); Bexxar® (Tositumomab); Bseron® (Interferon-β); bococizumab (anti-PCSK9 monoclonal antibody designated as L1L3, see U.S. Pat. No. 8,080,243); Campath® (Alemtuzumab); Dynepo® (Epoetin delta); Velcade® (bortezomib); MLN0002 (anti-a4 7 mAb); MLN1202 (anti-CCR2 chemokine receptor mAb); Enbrel® (etanercept); Eprex® (Epoetin alfa); Erbitux® (Cetuximab); evolocumab (anti-PCSK9 monoclonal antibody designated as 21B12, see U.S. Pat. No. 8,030,467); Genotropin® (Somatropin); Herceptin® (Trastuzumab); Humatrope® (somatropin [rDNA origin] for injection); Humira® (Adalimumab); Infergen® (Interferon Alfacon-1); Natrecor® (nesiritide); Kineret® (Anakinra), Leukine® (Sargamostim); LymphoCide® (Epratuzumab); Benlysta™ (Belimumab); Metalyse® (Tenecteplase); Mircera® (methoxy polyethylene glycol-epoetin β); Mylotarg® (Gemtuzumab ozogamicin); Raptiva® (efalizumab); Cimzia® (certolizumab pegol); Soliris™ (Eculizumab); Pexelizumab (Anti-C5 Complement); MEDI-524 (Numax®); Lucentis® (Ranibizumab); Edrecolomab (Panorex®); Trabio® (lerdelimumab); TheraCim hR3 (Nimotuzumab); Omnitarg (Pertuzumab, 2C4); Osidem® (IDM-I); OvaRex® (B43.13); Nuvion® (visilizumab); Cantuzumab mertansine (huC242-DMI); NeoRecormon® (Epoetin β); Neumega® (Oprelvekin); Neulasta® (Pegylated filgastrim, pegylated G-CSF, pegylated hu-Met-G-CSF); Neupogen® (Filgrastim); Orthoclone OKT3® (Muromonab-CD3), Procrit® (Epoetin alfa); Remicade® (Infliximab), Reopro® (Abciximab), Actemra® (anti-IL6 Receptor mAb), Avastin® (Bevacizumab), HuMax-CD4 (zanolimumab), Rituxan® (Rituximab); Tarceva® (Erlotinib); Roferon-A®-(Interferon alfa-2a); Simulect® (Basiliximab); Stelara™ (Ustekinumab); Prexige® (lumiracoxib); Synagis® (Palivizumab); 146B7-CHO (anti-IL15 antibody, see U.S. Pat. No. 7,153,507), Tysabri® (Natalizumab); Valortim® (MDX-1303, anti-B. anthracis Protective Antigen mAb); ABthrax™; Vectibix® (Panitumumab); Xolair® (Omalizumab), ETI211 (anti-M RSA mAb), IL-I Trap (the Fc portion of human IgG1 and the extracellular domains of both IL-I receptor components (the Type I receptor and receptor accessory protein)), VEGF Trap (lg domains of VEGFR1 fused to IgG1 Fc), Zenapax® (Daclizumab); Zenapax® (Daclizumab), Zevalin® (Ibritumomab tiuxetan), Zetia (ezetimibe), Atacicept (TACI-Ig), anti-a437-integrin mAb (vedolizumab); galiximab (anti-CD80 monoclonal antibody), anti-CD23 mAb (lumiliximab); BR2-Fc (huBR3/huFc fusion protein, soluble BAFF antagonist); Simponi (Golimumab); Mapatumumab (human anti-TRAIL Receptor-1 mAb); Ocrelizumab (anti-CD20 human mAb); HuMax-EGFR (zalutumumab); M200 (Volociximab, anti-a5 1 integrin mAb); MDX-010 (Ipilimumab, anti-CTLA-4 mAb and VEGFR-I (IMC-18F1); anti-BR3 mAb; anti-Clostridium difficile Toxin A and Toxin B C mAbs M DX-066 (CDA-I) and MDX-1388); anti-CD22 dsFv-PE38 conjugates (CAT-3888 and CAT-8015); anti-CD25 mAb (HuMax-TAC); Adecatumumab (MT201, anti-EpCAM-CD326 mAb); MDX-060, SGN-30, SGN-35 (anti-CD30 mAbs); M DX-1333 (anti-IFNAR); HuMax CD38 (anti-CD38 mAb); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); M EDI-545, MDX-1103 (anti-IFNa mAb); anti-IGFIR mAb; anti-IGF-IR mAb (HuMax-Inflam); anti-IL 12/IL23p40 mAb (Briakinumab); anti-IL-23pl9 mAb (LY2525623); anti-IL13 mAb (CAT-354); anti-IL-17 mAb (AlN457); anti-IL2Ra mAb (HuMax-TAC); anti-IL5 Receptor mAb; anti-integrin receptors mAb (MDX-018, CNTO 95); anti-IPIO Ulcerative Colitis mAb (MDX-1100); anti-LLY antibody; BMS-66513; anti-Mannose Receptor/hCG mAb (MDX-1307); anti-mesothelin dsFv-PE38 conjugate (CAT-5001); anti-PDImAb (M DX-1 106 (ONO-4538)); anti-PDGFRa antibody (IMC-3G3); anti-TGF mAb (GC-1008); anti-TRAIL Receptor-2 human mAb (HGS-ETR2); anti-TWEAK mAb; anti-VEGFR/FIt-1 mAb; anti-ZP3 mAb (HuMax-ZP3); NVS Antibody #1; and NVS Antibody #2.

Additional examples of antibodies (and antigen-binding fragments thereof) include; abagovomab, abciximab, actoxumab, adalimumab, afelimomab, afutuzumab, alacizumab, alacizumab pegol, ald518, alemtuzumab, alemtuzumab, alirocumab, altinumab, altumomab, amatuximab, anatumomab mafenatox, anrukinzumab, apolizumab, arcitumomab, aselizumab, atlizumab, atorolimiumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, cr6261, crenezumab, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enokizumab, enoticumab, enoticumab, ensituximab, epitumomab cituxetan, epratuzumab, erenumab, erlizumab, ertumaxomab, etaracizumab, etrolizumab, evolocumab, exbivirumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumoma tiuxetan, icrucumab, igovomab, imciromab, i mgatuzumab, inclacumab, indatuximab ravtansine, infliximab, inolimomab, inotuzumab ozogamicin, intetumumab, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, matuzumab, mavrilimumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, palivizumab, panitumumab, panobacumab, parsatuzuma pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, seviruma sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, tefibazumab, telimomab aritox, telimomab aritox, tenatumomab, tenatumomab, teneliximab, teplizumab, teprotumumab, TGN1412, ticilimumab, tigatuzumab, tildrakizumab, TNX-650, tocilizumab, tocilizumab, toralizumab, tositumomab, tralokinumab, trastuzumab, TRBS07, tregalizumab, tremelimumab, tremelimumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, and zolimomab aritox.

### Biomolecule Variant Library

In some embodiments, the methods described herein comprise the generation, expression and analysis of a biomolecule variant library. As used herein, the term “library” or “population” refers to a collection of at least two different molecules, such as nucleic acid sequences (e.g., genes, oligonucleotides, etc.) or expression products (e.g., enzymes or other proteins) therefrom. A library or population generally includes a number of different molecules. For example, a library or population typically includes at least about 10 different molecules. Large libraries typically include at least about 100 different molecules, more typically at least about 1000 different molecules. For some applications, the library includes at least about 10000 or more different molecules. In certain embodiments, the library contains a number variant or chimeric nucleic acids or proteins produced by a directed evolution procedure.

Directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,830,721, 6,132,970, 6,420,175, 6,277,638, 6,365,408, 6,602,986, 7,288,375, 6,287,861, 6,297,053, 6,576,467, 6,444,468, 5,811238, 6,117,679, 6,165,793, 6,180,406, 6,291,242, 6,995,017, 6,395,547, 6,506,602, 6,519,065, 6,506,603, 6,413,774, 6,573,098, 6,323,030, 6,344,356, 6,372,497, 7,868,138, 5,834,252, 5,928,905, 6,489,146, 6,096,548, 6,387,702, 6,391,552, 6,358,742, 6,482,647, 6,335,160, 6,653,072, 6,355,484, 6,03,344, 6,319,713, 6,613,514, 6,455,253, 6,579,678, 6,586,182, 6,406,855, 6,946,296, 7,534,564, 7,776,598, 5,837,458, 6,391,640, 6,309,883, 7,105,297, 7,795,030, 6,326,204, 6,251,674, 6,716,631, 6,528,311, 6,287,862, 6,335,198, 6,352,859, 6,379,964, 7,148,054, 7,629,170, 7,620,500, 6,365,377, 6,358,740, 6,406,910, 6,413,745, 6,436,675, 6,961,664, 7,430,477, 7,873,499, 7,702,464, 7,783,428, 7,747,391, 7,747,393, 7,751,986, 6,376,246, 6,426,224, 6,423,542, 6,479,652, 6,319,714, 6,521,453, 6,368,861, 7,421,347, 7,058,515, 7,024,312, 7,620,502, 7,853,410, 7,957,912, 7,904,249, and all related non-US counterparts; Ling et al., Anal. Biochem, 254 (2): 157-78 [1997]; Dale et al., Meth. Mol. Biol, 57:369-74 [1996]; Smith, Ann. Rev. Genet, 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol, 3:284-290 [1999]; Christians et al, Nat. BiotechnoL, 17:259-264 [1999]; Crameri et al, Nature, 391:288-291 [1998]; Crameri, et al, Nat. BiotechnoL, 15:436-438 [1997]; Zhang et al, Proc. Nat. Acad. Sci. U.S.A., 94:4504-4509 [1997]; Crameri et al, Nat. BiotechnoL, 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

In certain embodiments, directed evolution methods generate protein variant libraries by recombining genes encoding variants developed from a parent protein, as well as by recombining genes encoding variants in a parent protein variant library. Two nucleic acids are “recombined” when sequences from each of the two nucleic acids are combined in a progeny nucleic acid. Two sequences are “directly” recombined when both of the nucleic acids are substrates for recombination. The methods may employ oligonucleotides containing sequences or subsequences encoding at least one protein of a parental variant library. Some of the oligonucleotides of the parental variant library may be closely related, differing only in the choice of codons for alternate amino acids selected to be varied by recombination with other variants. The method may be performed for one or multiple cycles until desired results are achieved. If multiple cycles are used, each typically involves a screening step to identify those variants that have acceptable or improved performance and are candidates for use in at least one subsequent recombination cycle. In some embodiments, the screening step involves a virtual protein screening system for determining the catalytic activity and selectivity of enzymes for desired substrates.

In some embodiments, the variant sequences can be generated by CRISPR/Cas9-mediated homology-directed repair (HDR).

In some embodiments, directed evolution methods generate protein variants by site directed mutagenesis at defined residues. These defined residues are typically identified by structural analysis of binding sites, quantum chemistry analysis, sequence homology analysis, sequence activity models, etc. Some embodiments employ saturation mutagenesis, in which one tries to generate all possible (or as close to as possible) mutations at a specific site, or narrow region of a gene.

“Shuffling” and “gene shuffling” are types of directed evolution methods that recombine a collection of fragments of the parental polynucleotides through a series of chain extension cycles. In certain embodiments, one or more of the chain extension cycles is self-priming; i.e., performed without the addition of primers other than the fragments themselves. Each cycle involves annealing single stranded fragments through hybridization, subsequent elongation of annealed fragments through chain extension, and denaturing. Over the course of shuffling, a growing nucleic acid strand is typically exposed to multiple different annealing partners in a process sometimes referred to as “template switching,” which involves switching one nucleic acid domain from one nucleic acid with a second domain from a second nucleic acid (i.e., the first and second nucleic acids serve as templates in the shuffling procedure).

Template switching frequently produces chimeric sequences, which result from the introduction of crossovers between fragments of different origins. The crossovers are created through template switched recombinations during the multiple cycles of annealing, extension, and denaturing. Thus, shuffling typically leads to production of variant polynucleotide sequences. In some embodiments, the variant sequences comprise, a “library” of variants (i.e., a group comprising multiple variants). In some embodiments of these libraries, the variants contain sequence segments from two or more of parent polynucleotides. When two or more parental polynucleotides are employed, the individual parental polynucleotides are sufficiently homologous that fragments from different parents hybridize under the annealing conditions employed in the shuffling cycles. In some embodiments, the shuffling permits recombination of parent polynucleotides having relatively limited/low homology levels. Often, the individual parent polynucleotides have distinct and/or unique domains and/or other sequence characteristics of interest. When using parent polynucleotides having distinct sequence characteristics, shuffling can produce highly diverse variant polynucleotides.

Various shuffling techniques are known in the art. See e.g., U.S. Pat. Nos. 6,917,882, 7,776,598, 8,029,988, 7,024,312, and 7,795,030, all of which are incorporated herein by reference in their entireties.

Some directed evolution techniques employ “Gene Splicing by Overlap Extension” or “gene SOEing,” which is a PCR-based method of recombining DNA sequences without reliance on restriction sites and of directly generating mutated DNA fragments in vitro. In some implementations of the technique, initial PCRs generate overlapping gene segments that are used as template DNA for a second PCR to create a full-length product. Internal PCR primers generate overlapping, complementary 3′ ends on intermediate segments and introduce nucleotide substitutions, insertions or deletions for gene splicing. Overlapping strands of these intermediate segments hybridize at 3′ region in the second PCR and are extended to generate the full-length product. In various applications, the full length product is amplified by flanking primers that can include restriction enzyme sites for inserting the product into an expression vector for cloning purposes. See, e.g., Horton, et al, Biotechniques, 8(5): 528-35 [1990]. “Mutagenesis” is the process of introducing at least one mutation into a standard or reference sequence such as a parent nucleic acid or parent polypeptide. Site directed mutagenesis is one example of a useful technique for introducing mutations, although any suitable method finds use. Thus, alternatively or in addition, the mutants may be provided by gene synthesis, saturating random mutagenesis, semisynthetic combinatorial libraries of residues, recursive sequence recombination (“RSR”) (See e.g., US Patent Application Publ. No. 2006/0223143, incorporated by reference herein in its entirety), gene shuffling, error-prone PCR, and/or any other suitable method.

One example of a suitable saturation mutagenesis procedure is described in US Patent Application Publ. No. 2010/0093560, which is incorporated herein by reference in its entirety. A “fragment” is any portion of a sequence of nucleotides or amino acids.

In one embodiment of the disclosure, the antibody or antibody fragment variant library comprises about 10⁷ to about 10²⁰ different antibody variants and/or polynucleotide sequences encoding the antibody variants of the library. In some embodiments, the libraries of the instant disclosure are designed to include 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ different (i.e., unique) antibody variants and/or polynucleotide sequences encoding the antibody variants. In certain embodiments, the libraries of the disclosure may comprise or encode about 10³ to about 10⁵, about 10⁵ to about 10⁷, about 10⁷ to about 10⁹, about 10⁹ to about 10¹¹, about 10¹¹ to about 10¹³, about 10¹³ to about 10¹⁵, about 10¹⁵ to about 10¹⁷, or about 10¹⁷ to about 10²⁰ different antibody variants. In certain embodiments of the disclosure, the diversity of the libraries may be characterized as being greater than or less than one or more of the diversities enumerated herein, for example greater than about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ or less than about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰.

The genetic diversity of the host cell population can be defined as the number of different genetic variants present in the host cell population (e.g., biomolecule variant library), the number of different genetic variants relative to a negative control, and/or the number of different genetic variants relative to a reference cell strain. The number of genetic variants may be the actual number of variants or a calculated (“target”) number of genetic variants in the host cell population. These variants may be the result of one or more genetic (e.g., nucleic acid sequence) differences in the host cell genome between cells, one or more genetic (e.g., nucleic acid sequence) differences in expression construct(s) between host cells, or a combination thereof. In some examples, the genetic differences include alteration, deletion, or insertion of one or more nucleotides of a sequence or insertion or deletion of one or more elements (such as one or more tags, domains, expression control sequences, and/or associated proteins).

In some embodiments, the genetic diversity of the host cell population is at least 500, at least 1000, at least 2000, at least 5000, at least 10,000, and least 50,000, at least 100,000, at least 200,000, at least 500,000, at least 1,000,000, at least 2,000,000, at least 5,000,000, at least 10,000,000, at least 100,000,000, at least 500,000,000, or at least 1,000,000,000. In other examples, the genetic diversity is about 1000-1,000,000,000, such as about 1000-10,000, about 5000-50,000, about 50,000-200,000, about 100,000-500,000, about 200,000-1,000,000, about 500,000-2,000,000, about 1,000,000-5,000,000, about 5,000,000-50,000,000, about 20,000,000-100,000,000, about 50,000,000-500,000,000, or about 500,000,000-1,000,000,000.

Any type of genetic diversity can be probed using the methods provided herein. In some embodiments, the genetic diversity includes one or more of differences (including alteration or presence or absence) between a gene product of interest (including but not limited to coding sequence variants and codon-optimization), promoters (including constitutive and/or inducible promoters), chaperones, ribosome binding sequences, tags, nuclear localization signals, signal peptides, knockout or knockin of one or more genes, presence of one or more (such as 1, 2, 3, or more) plasmids, or any combination thereof. In some examples, the genetic diversity is generated by standard directed genetic modification techniques. In other examples, the genetic diversity is generated by random mutagenesis, error-prone PCR mutagenesis, or transposon mutagenesis (e.g., Tn5). A combination of techniques can also be used to generate additional levels of genetic diversity.

Additional methods for making alterations to host cell genomes or expression constructs in order to change nucleotide sequences and/or to eliminate, reduce, or change gene function are known in the art. Methods of making targeted disruptions of genes in host cells such as E. coli and other prokaryotes have been described (Muyrers et al., “Rapid modification of bacterial artificial chromosomes by ET-recombination”, Nucleic Acids Res 1999 Mar. 15; 27(6): 1555-1557; Datsenko and Wanner, “One-step inactivation of chromosomal genes in Escherichia coli K-12 using PCR products”, Proc Natl Acad Sci USA 2000 Jun. 6; 97(12): 6640-6645), and kits for using similar Red/ET recombination methods are commercially available (for example, the Quick & Easy E. coli Gene Deletion Kit from Gene Bridges GmbH, Heidelberg, Germany). Red/ET recombination methods can also be used to replace a promoter sequence with that of a different promoter, such as a constitutive promoter, or an artificial promoter that is predicted to promote a certain level of transcription (De Mey et al., “Promoter knock-in: a novel rational method for the fine tuning of genes”, BMC Biotechnol 2010 Mar. 24; 10:26). The function of host cell genomes or expression constructs can also be eliminated or reduced by RNA silencing methods (Man et al, “Artificial trans-encoded small non-coding RNAs specifically silence the selected gene expression in bacteria”, Nucleic Acids Res 2011 April; 39(8): e50, Epub 2011 Feb. 3). The Gibson assembly method (Gibson, “Enzymatic assembly of overlapping DNA fragments”, Methods Enzymol 2011; 498:349-361; doi: 10.1016/B978-0-12-385120-8.00015-2) can also be used to make targeted changes in host cell genomes or expression constructs, such as insertions, deletions, and point mutations. Another method for making directed alterations in host cell genomes or expression constructs utilizes CRISPR (clustered regularly interspaced short palindromic repeats) nucleotide sequences and Cas9 (CRISPR-associated protein 9), which recognizes and cleaves nucleotide sequences that are complementary to CRISPR sequences. Further, changes to host cell genomes can be introduced through traditional genetic methods.

### Host Cells

The methods described herein comprise expressing an antibody or an antigen binding protein in a host cell. A variety of host cells are suitable for expressing an antibody and these may be selected from, for example, prokaryotic cells, yeast cells, insects cells, mammalian cells or transgenic animals or plants. In one embodiment, the host cells are E. coli cells. As described herein, in another embodiment the SoluPro E. coli stain is contemplated (See, e.g., WO/2014/025663 and WO/2017/106583).

Non-limiting examples of suitable mammalian host cells include, but are not limited to, Chinese hamster ovary cells (CHO); monkey kidney CV1 cells transformed by SV440 (COS cells, COS-7, ATCC CRL-1651); human embryonic kidney cells (e.g., 293 cells); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells; human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); and mouse mammary tumor cells (MMT 060562, ATCC CCL-51).

In an embodiment, the host cell is prokaryotic. Prokaryotic cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Exemplary host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescens), and Shigella.

As described in International Publication No. WO 2017/106583, incorporated by reference in its entirety herein, producing an antigen binding protein at commercial scale and in soluble form is addressed by providing suitable host cells capable of growth at high cell density in fermentation culture, and which can produce soluble gene products in the oxidizing host cell cytoplasm through highly controlled inducible gene expression. Prokaryotic cells with these qualities are produced by combining some or all of the following characteristics: (1) The host cells are genetically modified to have an oxidizing cytoplasm, through increasing the expression or function of oxidizing polypeptides in the cytoplasm, and/or by decreasing the expression or function of reducing polypeptides in the cytoplasm. Specific examples of such genetic alterations are provided herein. Optionally, host cells can also be genetically modified to express chaperones and/or cofactors that assist in the production of the desired gene product(s), and/or to glycosylate polypeptide gene products. (2) The host cells comprise one or more expression constructs designed for the expression of one or more gene products of interest; in certain embodiments, at least one expression construct comprises an inducible promoter and a polynucleotide encoding a gene product to be expressed from the inducible promoter. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araE, araG, araH, rhaT, xyIF, xyIG, and xyIH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one said inducible promoter is selected from the group consisting of araA, araB, araD, prpB, prpD, rhaA, rhaB, rhaD, xylA, and xyIB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmIA, rmIB, rmIC, and rmID.

Prokaryotic Cells with Oxidizing Cytoplasm. Examples of host cells are provided that allow for the efficient and cost-effective expression of gene products, including components of multimeric products. Host cells can include, in addition to isolated cells in culture, cells that are part of a multicellular organism, or cells grown within a different organism or system of organisms. In certain embodiments of the disclosure, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli (B strain) EB0001 cells (also called E. coli ASE (DGH) cells), or are E. coli (B strain) EB0002 cells. In growth experiments with E. coli host cells having oxidizing cytoplasm, specifically the E. coli B strains SHuffle® Express (NEB Catalog No. C3028H) and SHuffle® T7 Express (NEB Catalog No. C3029H) and the E. coli K strain SHuffle® T7 (NEB Catalog No. C3026H), these E. coli B strains with oxidizing cytoplasm are able to grow to much higher cell densities than the most closely corresponding E. coli K strain (International Publication No. WO 2017/106583).

Certain alterations can be made to the gene functions of host cells comprising inducible expression constructs, to promote efficient and homogeneous induction of the host cell population by an inducer. In some embodiments, the combination of expression constructs, host cell genotype, and induction conditions results in at least 75% (more preferably at least 85%, and most preferably, at least 95%) of the cells in the culture expressing gene product from each induced promoter, as measured by the method of Khlebnikov et al. described in Example 9 of International Publication No. WO 2017/106583. For host cells other than E. coli, these alterations can involve the function of genes that are structurally similar to an E. coli gene, or genes that carry out a function within the host cell similar to that of the E. coli gene. Alterations to host cell gene functions include eliminating or reducing gene function by deleting the gene protein-coding sequence in its entirety, or deleting a large enough portion of the gene, inserting sequence into the gene, or otherwise altering the gene sequence so that a reduced level of functional gene product is made from that gene. Alterations to host cell gene functions also include increasing gene function by, for example, altering the native promoter to create a stronger promoter that directs a higher level of transcription of the gene, or introducing a missense mutation into the protein-coding sequence that results in a more highly active gene product. Alterations to host cell gene functions include altering gene function in any way, including for example, altering a native inducible promoter to create a promoter that is constitutively activated. In addition to alterations in gene functions for the transport and metabolism of inducers, as described herein with relation to inducible promoters, and/or an altered expression of chaperone proteins, it is also possible to alter the reduction-oxidation environment of the host cell.

Host cell reduction-oxidation environment. In bacterial cells such as E. coli, proteins that need disulfide bonds are typically exported into the periplasm where disulfide bond formation and isomerization is catalyzed by the Dsb system, comprising DsbABCD and DsbG. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., Microb Cell Fact 2011 May 14; 10:32). It is also possible to express cytoplasmic forms of these Dsb proteins, such as a cytoplasmic version of DsbA and/or of DsbC (′cDsbA or ‘cDsbC’), that lacks a signal peptide and therefore is not transported into the periplasm. Cytoplasmic Dsb proteins such as cDsbA and/or cDsbC are useful for making the cytoplasm of the host cell more oxidizing and thus more conducive to the formation of disulfide bonds in heterologous proteins produced in the cytoplasm. The host cell cytoplasm can also be made less reducing and thus more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductants, such as dithiothreitol (DTT). Suppressor mutations (such as ahpC* and ahpCA, Lobstein et al., Microb Cell Fact 2012 May 8; 11:56; doi: 10.1186/1475-2859-11-56) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dupl62-169 (Faulkner et al., Proc Natl Acad Sci USA 2008 May 6; 105 (18): 6735-6740, Epub 2008 May 2). In such strains with oxidizing cytoplasm, exposed protein cysteines become readily oxidized in a process that is catalyzed by thioredoxins, in a reversal of their physiological function, resulting in the formation of disulfide bonds. Other proteins that may be helpful to reduce the oxidative stress effects in host cells of an oxidizing cytoplasm are HPI (hydroperoxidase I) catalase-peroxidase encoded by E. coli katG and HPII (hydroperoxidase II) catalase-peroxidase encoded by E. coli katE, which disproportionate peroxide into water and 02 (Farr and Kogoma, Microbiol Rev. 1991 December; 55 (4): 561-585; Review). Increasing levels of KatG and/or KatE protein in host cells through induced coexpression or through elevated levels of constitutive expression is an aspect of some embodiments of the disclosure.

Another alteration that can be made to host cells is to express the sulfhydryl oxidase Ervlp from the inner membrane space of yeast mitochondria in the host cell cytoplasm, which has been shown to increase the production of a variety of complex, disulfide-bonded proteins of eukaryotic origin in the cytoplasm of E. coli, even in the absence of mutations in gor or trxB (Nguyen et al, Microb Cell Fact 2011 Jan. 7; 10:1).

Host cells comprising expression constructs preferably also express cDsbA and/or cDsbC and/or Ervlp; are deficient in trxB gene function; are also deficient in the gene function of either gor, gshB, or gshA; optionally have increased levels of katG and/or katE gene function; and express an appropriate mutant form of AhpC so that the host cells can be grown in the absence of DTT.

Chaperones. In some embodiments, desired gene products are coexpressed with other gene products, such as chaperones, that are beneficial to the production of the desired gene product. Chaperones are proteins that assist the non-covalent folding or unfolding, and/or the assembly or disassembly, of other gene products, but do not occur in the resulting monomeric or multimeric gene product structures when the structures are performing their normal biological functions (having completed the processes of folding and/or assembly). Chaperones can be expressed from an inducible promoter or a constitutive promoter within an expression construct, or can be expressed from the host cell chromosome; preferably, expression of chaperone protein(s) in the host cell is at a sufficiently high level to produce coexpressed gene products that are properly folded and/or assembled into the desired product. Examples of chaperones present in E. coli host cells are the folding factors DnaK/DnaJ/GrpE, DsbC/DsbG, GroEL/GroES, IbpA/lbpB, Skp, Tig (trigger factor), and FkpA, which have been used to prevent protein aggregation of cytoplasmic or periplasmic proteins. DnaK/DnaJ/GrpE, GroEL/GroES, and ClpB can function synergistically in assisting protein folding and therefore expression of these chaperones in combinations has been shown to be beneficial for protein expression (Makino et al., Microb Cell Fact 2011 May 14; 10:32). When expressing eukaryotic proteins in prokaryotic host cells, a eukaryotic chaperone protein, such as protein disulfide isomerase (PDI) from the same or a related eukaryotic species, is in certain embodiments of the disclosure coexpressed or inducibly coexpressed with the desired gene product.

One chaperone that can be expressed in host cells is a protein disulfide isomerase from Humicola insolens, a soil hyphomycete (soft-rot fungus). An amino acid sequence of Humicola insolens PDI is shown as SEQ ID NO: 1 of International Publication No. WO 2017/106583; it lacks the signal peptide of the native protein so that it remains in the host cell cytoplasm. The nucleotide sequence encoding PDI was optimized for expression in E. coli; the expression construct for PDI is shown as SEQ ID NO: 2 of International Publication No. WO 2017/106583. SEQ ID NO: 2 contains a GCTAGC Nhel restriction site at its 5′ end, an AGGAGG ribosome binding site at nucleotides 7 through 12, the PDI coding sequence at nucleotides 21 through 1478, and a GTCGAC Sail restriction site at its 3′ end. The nucleotide sequence of SEQ ID NO: 2 was designed to be inserted immediately downstream of a promoter, such as an inducible promoter. The Nhel and Sail restriction sites in SEQ ID NO: 2 can be used to insert it into a vector multiple cloning site, such as that of the pSOL expression vector (SEQ ID NO: 3 of International Publication No. WO 2017/106583), described in published US patent application US 2015/353940A1, which is incorporated by reference in its entirety herein. Other PDI polypeptides can also be expressed in host cells, including PDI polypeptides from a variety of species (Saccharomyces cerevisiae (UniProtKB PI 7967), Homo sapiens (UniProtKB P07237), Mus musculus (UniProtKB P09103), Caenorhabditis elegans (UniProtKB Q 17770 and Q 17967), Arabdopsis thaliana (UniProtKB 048773, Q9X101, Q9S G3, Q9LJU2, Q9MAU6, Q94F09, and Q9T042), Aspergillus niger (UniProtKB Q12730) and also modified forms of such PDI polypeptides. In certain embodiments of the disclosure, a PDI polypeptide expressed in host cells of the disclosure shares at least 70%, or 80%, or 90%, or 95% amino acid sequence identity across at least 50% (or at least 60%, or at least 70%, or at least 80%, or at least 90%) of the length of SEQ ID NO: 1 of International Publication No. WO 2017/106583, where amino acid sequence identity is determined according to Example 10 of International Publication No. WO 2017/106583.

Cellular transport of cofactors. Common cofactors include ATP, coenzyme A, flavin adenine dinucleotide (FAD), NAD+/NADH, and heme. Polynucleotides encoding cofactor transport polypeptides and/or cofactor synthesizing polypeptides can be introduced into host cells, and such polypeptides can be constitutively expressed, or inducibly coexpressed with the gene products to be produced by methods of the disclosure.

Glycosylation of polypeptide gene products. Host cells can have alterations in their ability to glycosylate polypeptides. For example, eukaryotic host cells can have eliminated or reduced gene function in glycosyltransferase and/or oligo saccharyltransferase genes, impairing the normal eukaryotic glycosylation of polypeptides to form glycoproteins.

Prokaryotic host cells such as E. coli, which do not normally glycosylate polypeptides, can be altered to express a set of eukaryotic and prokaryotic genes that provide a glycosylation function (DeLisa et al., WO 2009/089154A2, 2009 Jul. 16).

Available host cell strains with altered gene functions. To create preferred strains of host cells to be used in the expression systems and methods of the disclosure, it is useful to start with a strain that already comprises desired genetic alterations (Table A; International Publication No. WO 2017/106583).

TABLE A
Exemplary host cell strains

| Strain | Genotype | Source |
|---|---|---|
| E, coli TOP10 | F- mcrA Δ(mrr-hsdRMS-mcrBC) | Invitrogen Life Technologies |
| | Φ80lacZ ΔM15 Δlacx74 recA1 araD139 | Catalog Nos. C404-10, |
| | Δ(ara-leu)7697 galU galK rpsL (Str^{R}) | C4040-03, C4040-06, |
| | endA1 nupG λ | C4040-50, and C4040-52 |
| E, coli Origami ™ | Δ(ara-leu)7697 Δlacx74 ΔphoA Pvull | Merck (EMD Millipore |
| 2 | phoR araD139 ahpC gale galk rpsL | Chemicals) Catalog No. |
| | F′[lac⁺ lacl^{q} pro] gor522::Tn10 trxB | 71344 |
| | (Str^{R} Tet^{R}) | |
| E, coli SHuffle ® | fhuA2 [Ion] ompT ahpC gal λatt::pNEB3- | New England Biolabs |
| Express | r1-cDsbc (Spec, lacl) ΔtrxB sulA11 | Catalog No. C3028H |
| | R(mcr-73::miniTn10-Tet^{S})2 [dcm] | |
| | R(zgb-210::Tn10-Tet^{S}) endA1 Δgor | |
| | Δ(mcrC-mrr)114::IS10 | |

### Expression Constructs

In some embodiments, a prokaryotic cell described herein comprises one or more expression constructs that may optionally include one or more inducible promoters to express an antigen binding protein of interest.

The term “expression construct” as used herein refers to polynucleotides designed for the expression of one or more antigen binding proteins of interest, and thus are not naturally occurring molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more antigen binding proteins. One or more expression constructs can be integrated into a host cell chromosome or be maintained on an extrachromosomal polynucleotide such as a plasmid or artificial chromosome. The following are descriptions of particular types of polynucleotide sequences that can be used in expression constructs for the expression or coexpression of gene products, including fusion proteins as described herein.

Origins of replication. Expression constructs must comprise an origin of replication, also called a replicon, in order to be maintained within the host cell as independently replicating polynucleotides. Different replicons that use the same mechanism for replication cannot be maintained together in a single host cell through repeated cell divisions. As a result, plasmids can be categorized into incompatibility groups depending on the origin of replication that they contain, as shown in Table 2 of International Publication No. WO 2016/205570. Origins of replication can be selected for use in expression constructs on the basis of incompatibility group, copy number, and/or host range, among other criteria. As described above, if two or more different expression constructs are to be used in the same host cell for the coexpression of multiple gene products, it is best if the different expression constructs contain origins of replication from different incompatibility groups: a pMBI replicon in one expression construct and a pl5A replicon in another, for example. The average number of copies of an expression construct in the cell, relative to the number of host chromosome molecules, is determined by the origin of replication contained in that expression construct. Copy number can range from a few copies per cell to several hundred (Table 2 of WO/2016/205570). In some embodiments, different expression constructs are used which comprise inducible promoters that are activated by the same inducer, but which have different origins of replication. By selecting origins of replication that maintain each different expression construct at a certain approximate copy number in the cell, it is possible to adjust the levels of overall production of a gene product expressed from one expression construct, relative to another gene product expressed from a different expression construct. As an example, to coexpress subunits A and B of a multimeric protein, an expression construct is created which comprises the colEl replicon, the am promoter, and a coding sequence for subunit A expressed from the am promoter: 'colEl-Para-A.

Another expression construct is created comprising the pl 5A replicon, the am promoter, and a coding sequence for subunit B: ‘pl5A-Para-B’. These two expression constructs can be maintained together in the same host cells, and expression of both subunits A and B is induced by the addition of one inducer, arabinose, to the growth medium. If the expression level of subunit A needed to be significantly increased relative to the expression level of subunit B, in order to bring the stoichiometric ratio of the expressed amounts of the two subunits closer to a desired ratio, for example, a new expression construct for subunit A could be created, having a modified pMB 1 replicon as is found in the origin of replication of the pUC9 plasmid (‘pUC9ori’): pUC9ori-Para-A. Expressing subunit A from a high-copy-number expression construct such as pUC9ori-Para-A should increase the amount of subunit A produced relative to expression of subunit B from pl5A-Para-B. In a similar fashion, use of an origin of replication that maintains expression constructs at a lower copy number, such as pSOOI (WO/2016/205570), could reduce the overall level of a gene product expressed from that construct. Selection of an origin of replication can also determine which host cells can maintain an expression construct comprising that replicon. For example, expression constructs comprising the colEl origin of replication have a relatively narrow range of available hosts, species within the Enterobacteriaceae family, while expression constructs comprising the RK2 replicon can be maintained in E. coli, Pseudomonas aeruginosa, Pseudomonas putida, Azotobacter vinelandii, and Alcaligenes eutrophus, and if an expression construct comprises the RK2 replicon and some regulator genes from the RK2 plasmid, it can be maintained in host cells as diverse as Sinorhizobium meliloti, Agrobacterium tumefaciens, Caulobacter crescentus, Acinetobacter calcoaceticus, and Rhodobacter sphaeroides (Kiies and Stahl, Microbiol Rev 1989 December; 53 (4): 491-516).

Similar considerations can be employed to create expression constructs for inducible expression or coexpression in eukaryotic cells. For example, the 2-micron circle plasmid of Saccharomyces cerevisiae is compatible with plasmids from other yeast strains, such as pSRI (ATCC Deposit Nos. 48233 and 66069; Araki et al., J Mol Biol 1985 Mar. 20; 182 (2): 191-203) and pKDI (ATCC Deposit No. 37519; Chen et al, Nucleic Acids Res 1986 Jun. 11; 14 (11): 4471-4481).

In some embodiments, the expression construct comprises a selection gene. A “selection gene”, also termed a selectable marker, encodes a protein necessary for the survival or growth of a host cell in a selective culture medium. Host cells not containing the expression construct comprising the selection gene will not survive in the culture medium. Typical selection genes encode proteins that confer resistance to antibiotics or other toxins, or that complement auxotrophic deficiencies of the host cell. One example of a selection scheme utilizes a drug such as an antibiotic to arrest growth of a host cell. Those cells that contain an expression construct comprising the selectable marker produce a protein conferring drug resistance and survive the selection regimen. Some examples of antibiotics that are commonly used for the selection of selectable markers (and abbreviations indicating genes that provide antibiotic resistance phenotypes) are: ampicillin (AmpR), chloramphenicol (CmIR or CmR), kanamycin (KanR), spectinomycin (SpcR), streptomycin (StrR), and tetracycline (TetR). Many of the plasmids in Table 2 of WO/2016/205570 comprise selectable markers, such as pBR322 (AmpR, TetR); pMOB45 (CmR, TetR); pACYCIW (AmpR, KanR); and pGBMI (SpcR, StrR). The native promoter region for a selection gene is usually included, along with the coding sequence for its gene product, as part of a selectable marker portion of an expression construct. Alternatively, the coding sequence for the selection gene can be expressed from a constitutive promoter.

Exemplary selectable markers include, but are not limited to, neomycin phosphotransferase (npt II), hygromycin phosphotransferase (hpt), dihydrofolate reductase (dhfr), zeocin, phleomycin, bleomycin resistance gene (ble), gentamicin acetyltransferase, streptomycin phosphotransferase, mutant form of acetolactate synthase (als), bromoxynil nitrilase, phosphinothricin acetyltransferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2), orotidine-5′-phosphate (OMP) decarboxylase (ura3), and orthologs of any of the foregoing.

Inducible promoter. As described herein, there are several different inducible promoters that can be included in expression constructs as part of the inducible coexpression systems of the disclosure. In some embodiments, inducible promoters share at least 80% polynucleotide sequence identity (more preferably, at least 90% identity, and most preferably, at least 95% identity) to at least 30 (more preferably, at least 40, and most preferably, at least 50) contiguous bases of a promoter polynucleotide sequence as defined in Table 1 of International Publication No. WO 2016/205570 by reference to the E. coli K-12 substrain MG1655 genomic sequence, where percent polynucleotide sequence identity is determined using the methods of Example 11 of WO/2016/205570. Under ‘standard’ inducing conditions (see Example 5 of International Publication No. WO 2016/205570), preferred inducible promoters have at least 75% (more preferably, at least 100%, and most preferably, at least 110%) of the strength of the corresponding ‘wild-type’ inducible promoter of E. coli K-12 substrain MG1655, as determined using the quantitative PCR method of De Mey et al. (Example 6 of International Publication No. WO 2016/205570). Within the expression construct, an inducible promoter is placed 5′ to (or ‘upstream of) the coding sequence for the gene product that is to be inducibly expressed, so that the presence of the inducible promoter will direct transcription of the gene product coding sequence in a 5’ to 3′ direction relative to the coding strand of the polynucleotide encoding the gene product.

Ribosome binding site. For polypeptide gene products, the nucleotide sequence of the region between the transcription initiation site and the initiation codon of the coding sequence of the gene product that is to be inducibly expressed corresponds to the 5′ untranslated region (‘UTR’) of the mRNA for the polypeptide gene product. Preferably, the region of the expression construct that corresponds to the 5′ UT comprises a polynucleotide sequence similar to the consensus ribosome binding site (RBS, also called the Shine-Dalgarno sequence) that is found in the species of the host cell. In prokaryotes (archaea and bacteria), the RBS consensus sequence is GGAGG or GGAGGU, and in bacteria such as E. coli, the RBS consensus sequence is AGGAGG or AGGAGGU. The RBS is typically separated from the initiation codon by 5 to 10 intervening nucleotides. In expression constructs, the RBS sequence is preferably at least 55% identical to the AGGAGGU consensus sequence, more preferably at least 70% identical, and most preferably at least 85% identical, and is separated from the initiation codon by 5 to 10 intervening nucleotides, more preferably by 6 to 9 intervening nucleotides, and most preferably by 6 or 7 intervening nucleotides. The ability of a given RBS to produce a desirable translation initiation rate can be calculated at the website salis.psu.edu/software/RBSLibraryCalculatorSearchMode, using the RBS Calculator; the same tool can be used to optimize a synthetic RBS for a translation rate across a 100,000+ fold range (Salis, Methods Enzymol 2011; 498:19-42).

Multiple cloning site. A multiple cloning site (MCS), also called a polylinker, is a polynucleotide that contains multiple restriction sites in close proximity to or overlapping each other. The restriction sites in the MCS typically occur once within the MCS sequence, and preferably do not occur within the rest of the plasmid or other polynucleotide construct, allowing restriction enzymes to cut the plasmid or other polynucleotide construct only within the MCS. Examples of MCS sequences are those in the pBAD series of expression vectors, including pBAD18, pBAD18-Cm, pBAD18-Kan, pBAD24, pBAD28, pBAD30, and pBAD33 (Guzman et al., J Bacteriol 1995 July; 177 (14): 4121-4130); or those in the pPRO series of expression vectors derived from the pBAD vectors, such as pPR018, pPR018-Cm, pPR018-Kan, pPR024, pPRO30, and pPR033 (U.S. Pat. No. 8,178,338 B2; May 15, 2012; Keasling, Jay). A multiple cloning site can be used in the creation of an expression construct: by placing a multiple cloning site 3′ to (or downstream of) a promoter sequence, the MCS can be used to insert the coding sequence for a gene product to be expressed or coexpressed into the construct, in the proper location relative to the promoter so that transcription of the coding sequence will occur. Depending on which restriction enzymes are used to cut within the MCS, there may be some part of the MCS sequence remaining within the expression construct after the coding sequence or other polynucleotide sequence is inserted into the expression construct. Any remaining MCS sequence can be upstream or, or downstream of, or on both sides of the inserted sequence. A ribosome binding site can be placed upstream of the MCS, preferably immediately adjacent to or separated from the MCS by only a few nucleotides, in which case the RBS would be upstream of any coding sequence inserted into the MCS. Another alternative is to include a ribosome binding site within the MCS, in which case the choice of restriction enzymes used to cut within the MCS will determine whether the RBS is retained, and in what relation to, the inserted sequences. A further alternative is to include a RBS within the polynucleotide sequence that is to be inserted into the expression construct at the MCS, preferably in the proper relation to any coding sequences to stimulate initiation of translation from the transcribed messenger RNA.

Expression from constitutive promoters. Expression constructs of the disclosure can also comprise coding sequences that are expressed from constitutive promoters. Unlike inducible promoters, constitutive promoters initiate continual gene product production under most growth conditions. One example of a constitutive promoter is that of the Tn3 bla gene, which encodes beta-lactamase and is responsible for the ampicillin-resistance (AmpR) phenotype conferred on the host cell by many plasmids, including pBR322 (ATCC 31344), pACYCIW (ATCC 37031), and pBAD24 (ATCC 87399). Another constitutive promoter that can be used in expression constructs is the promoter for the E. coli lipoprotein gene, Ipp, which is located at positions 1755731-1755406 (plus strand) in E. coli K-12 substrain MG1655 (Inouye and Inouye, Nucleic Acids Res 1985 May 10; 13 (9): 3101-3110). A further example of a constitutive promoter that has been used for heterologous gene expression in E. coli is the trpLEDCBA promoter, located at positions 1321169-1321133 (minus strand) in E. coli K-12 substrain MG1655 (Windass et al., Nucleic Acids Res 1982 Nov. 11; 10(21): 6639-6657). Constitutive promoters can be used in expression constructs for the expression of selectable markers, as described herein, and also for the constitutive expression of other gene products useful for the coexpression of the desired product. For example, transcriptional regulators of the inducible promoters, such as AraC, PrpR, RhaR, and XyIR, if not expressed from a bidirectional inducible promoter, can alternatively be expressed from a constitutive promoter, on either the same expression construct as the inducible promoter they regulate, or a different expression construct. Similarly, gene products useful for the production or transport of the inducer, such as PrpEC, AraE, or Rha, or proteins that modify the reduction-oxidation environment of the cell, as a few examples, can be expressed from a constitutive promoter within an expression construct. Gene products useful for the production of coexpressed gene products, and the resulting desired product, also include chaperone proteins, cofactor transporters, etc.

Signal Peptides. Polypeptide gene products expressed or coexpressed by the methods of the disclosure can contain signal peptides or lack them, depending on whether it is desirable for such gene products to be exported from the host cell cytoplasm into the periplasm, or to be retained in the cytoplasm, respectively. Signal peptides (also termed signal sequences, leader sequences, or leader peptides) are characterized structurally by a stretch of hydrophobic amino acids, approximately five to twenty amino acids long and often around ten to fifteen amino acids in length, that has a tendency to form a single alpha-helix. This hydrophobic stretch is often immediately preceded by a shorter stretch enriched in positively charged amino acids (particularly lysine). Signal peptides that are to be cleaved from the mature polypeptide typically end in a stretch of amino acids that is recognized and cleaved by signal peptidase. Signal peptides can be characterized functionally by the ability to direct transport of a polypeptide, either co-translationally or post-translationally, through the plasma membrane of prokaryotes (or the inner membrane of gram negative bacteria like E. coli), or into the endoplasmic reticulum of eukaryotic cells. The degree to which a signal peptide enables a polypeptide to be transported into the periplasmic space of a host cell like E. coli, for example, can be determined by separating periplasmic proteins from proteins retained in the cytoplasm, using a method such as described in Example 12 of International Publication No. WO 2016/205570.

Examples of inducible promoters and related genes are, unless otherwise specified, from Escherichia coli (E. coli) strain MG1655 (American Type Culture Collection deposit ATCC 700926), which is a substrain of E. coli K-12 (American Type Culture Collection deposit ATCC 10798). Table 1 of International Publication No. WO 2016/205570 lists the genomic locations, in E. coli MG1655, of the nucleotide sequences for these examples of inducible promoters and related genes. Nucleotide and other genetic sequences, referenced by genomic location as in Table 1 of International Publication No. WO 2016/205570, are expressly incorporated by reference herein. Additional information about E. coli promoters, genes, and strains described herein can be found in many public sources, including the online EcoliWiki resource, located at ecoliwiki.net.

Arabinose promoter. (As used herein, ‘arabinose’ means L-arabinose.) Several E. coli operons involved in arabinose utilization are inducible by arabinose-araBAD, araC, arciE, and araFGH—but the terms ‘arabinose promoter’ and ‘ara promoter’ are typically used to designate the araBAD promoter. Several additional terms have been used to indicate the E. coli araBAD promoter, such as Para, ParaB, ParaBAD, and PBAD—The use herein of ‘ara promoter’ or any of the alternative terms given above, means the E. coli araBAD promoter. As can be seen from the use of another term, ‘araC-araBAD promoter’, the araBAD promoter is considered to be part of a bidirectional promoter, with the araBAD promoter controlling expression of the araBAD operon in one direction, and the araC promoter, in close proximity to and on the opposite strand from the araBAD promoter, controlling expression of the araC coding sequence in the other direction. The AraC protein is both a positive and a negative transcriptional regulator of the araBAD promoter. In the absence of arabinose, the AraC protein represses transcription from PBAD, but in the presence of arabinose, the AraC protein, which alters its conformation upon binding arabinose, becomes a positive regulatory element that allows transcription from PBAD—The araBAD operon encodes proteins that metabolize L-arabinose by converting it, through the intermediates L-ribulose and L-ribulose-phosphate, to D-xylulose-5-phosphate. For the purpose of maximizing induction of expression from an arabinose-inducible promoter, it is useful to eliminate or reduce the function of AraA, which catalyzes the conversion of L-arabinose to L-ribulose, and optionally to eliminate or reduce the function of at least one of AraB and AraD, as well. Eliminating or reducing the ability of host cells to decrease the effective concentration of arabinose in the cell, by eliminating or reducing the cell's ability to convert arabinose to other sugars, allows more arabinose to be available for induction of the arabinose-inducible promoter. The genes encoding the transporters which move arabinose into the host cell are araE, which encodes the low-affinity L-arabinose proton symporter, and the araFGH operon, which encodes the subunits of an ABC superfamily high-affinity L-arabinose transporter. Other proteins which can transport L-arabinose into the cell are certain mutants of the LacY lactose permease: the LacY (AIWC) and the LacY (AIWV) proteins, having a cysteine or a valine amino acid instead of alanine at position 177, respectively (Morgan-Kiss et al., Proc Natl Acad Sci USA 2002 May 28; 99 (11): 7373-7377). In order to achieve homogeneous induction of an arabinose-inducible promoter, it is useful to make transport of arabinose into the cell independent of regulation by arabinose. This can be accomplished by eliminating or reducing the activity of the AraFGH transporter proteins and altering the expression of araE so that it is only transcribed from a constitutive promoter. Constitutive expression of araE can be accomplished by eliminating or reducing the function of the native araE gene, and introducing into the cell an expression construct which includes a coding sequence for the AraE protein expressed from a constitutive promoter. Alternatively, in a cell lacking AraFGH function, the promoter controlling expression of the host cell's chromosomal araE gene can be changed from an arabinose-inducible promoter to a constitutive promoter. In similar manner, as additional alternatives for homogenous induction of an arabinose-inducible promoter, a host cell that lacks AraE function can have any functional AraFGH coding sequence present in the cell expressed from a constitutive promoter. As another alternative, it is possible to express both the araE gene and the araFGH operon from constitutive promoters, by replacing the native araE and araFGH promoters with constitutive promoters in the host chromosome. It is also possible to eliminate or reduce the activity of both the AraE and the AraFGH arabinose transporters, and in that situation to use a mutation in the LacY lactose permease that allows this protein to transport arabinose. Since expression of the lacY gene is not normally regulated by arabinose, use of a LacY mutant such as LacY (A177C) or LacY (A177V), will not lead to the ‘all or none’ induction phenomenon when the arabinose-inducible promoter is induced by the presence of arabinose. Because the LacY (A177C) protein appears to be more effective in transporting arabinose into the cell, use of polynucleotides encoding the LacY (A177C) protein is preferred to the use of polynucleotides encoding the LacY (A177V) protein.

Propionate promoter. The ‘propionate promoter’ or ‘prp promoter’ is the promoter for the E. coli prpBCDE operon. Like the ara promoter, the prp promoter is part of a bidirectional promoter, controlling expression of the prpBCDE operon in one direction, and with the prpR promoter controlling expression of the prpR coding sequence in the other direction. The PrpR protein is the transcriptional regulator of the prp promoter, and activates transcription promoter, and activates transcription from the prp promoter when the PrpR protein binds 2-methylcitrate (‘2-MC’). Propionate (also called propanoate) is the ion, CH3CH2COO—, of propionic acid (or ‘propanoic acid’), and is the smallest of the ‘fatty’ acids having the general formula H(CH2)″COOH that shares certain properties of this class of molecules: producing an oily layer when salted out of water and having a soapy potassium salt. Commercially available propionate is generally sold as a monovalent cation salt of propionic acid, such as sodium propionate (CH3CH2COONa), or as a divalent cation salt, such as calcium propionate (Ca(CH3CH2COO)2). Propionate is membrane-permeable and is metabolized to 2-MC by conversion of propionate to propionyl-CoA by PrpE (propionyl-CoA synthetase), and then conversion of propionyl-CoA to 2-MC by PrpC (2-methylcitrate synthase). The other proteins encoded by the prpBCDE operon, PrpD (2-methylcitrate dehydratase) and PrpB (2-methylisocitrate lyase), are involved in further catabolism of 2-MC into smaller products such as pyruvate and succinate. In order to maximize induction of a propionate-inducible promoter by propionate added to the cell growth medium, it is therefore desirable to have a host cell with PrpC and PrpE activity, to convert propionate into 2-MC, but also having eliminated or reduced PrpD activity, and optionally eliminated or reduced PrpB activity as well, to prevent 2-MC from being metabolized. Another operon encoding proteins involved in 2-MC biosynthesis is the scpA-argK-scpBC operon, also called the sbm-yg/DGH operon. These genes encode proteins required for the conversion of succinate to propionyl-CoA, which can then be converted to 2-MC by PrpC. Elimination or reduction of the function of these proteins would remove a parallel pathway for the production of the 2-MC inducer, and thus might reduce background levels of expression of a propionate-inducible promoter, and increase sensitivity of the propionate-inducible promoter to exogenously supplied propionate. It has been found that a deletion of sbm-ygfD-ygfG-ygfH-ygfl, introduced into E. coli BL21 (DE3) to create strain JSB (Lee and Keasling, “A propionate-inducible expression system for enteric bacteria”, Appl Environ Microbiol 2005 November; 71 (11): 6856-6862), was helpful in reducing background expression in the absence of exogenously supplied inducer, but this deletion also reduced overall expression from the prp promoter in strain JSB. It should be noted, however, that the deletion sbm-ygfD-ygfG-ygfH-ygfl also apparently affects ygfl, which encodes a putative LysR-family transcriptional regulator of unknown function. The genes sbm-yg/DGH are transcribed as one operon, and ygfl is transcribed from the opposite strand. The 3′ ends of the ygfti and ygfl coding sequences overlap by a few base pairs, so a deletion that takes out all of the sbm-yg/DGH operon apparently takes out ygfl coding function as well. Eliminating or reducing the function of a subset of the sbm-ygfDGH gene products, such as YgfG (also called ScpB, methylmalonyl-CoA decarboxylase), or deleting the majority of the sbm-yg/DGH (or scpA-argK-scpBC) operon while leaving enough of the 3′ end of the ygfli (or scpC) gene so that the expression of ygfl is not affected, could be sufficient to reduce background expression from a propionate-inducible promoter without reducing the maximal level of induced expression.

Rhamnose promoter. (As used herein, ‘rhamnose’ means L-rhamnose.) The ‘rhamnose promoter’ or ‘rha promoter’, or PrhaSR, is the promoter for the E. coli rhaSR operon. Like the ara and prp promoters, the rha promoter is part of a bidirectional promoter, controlling expression of the rhaSR operon in one direction, and with the rhaBAD promoter controlling expression of the rhaBAD operon in the other direction. The rha promoter, however, has two transcriptional regulators involved in modulating expression: RhaR and RhaS. The RhaR protein activates expression of the rhaSR operon in the presence of rhamnose, while RhaS protein activates expression of the L-rhamnose catabolic and transport operons, rhaBAD and rhaT, respectively (Wickstrum et al, J Bacteriol 2010 January; 192(1): 225-232). Although the RhaS protein can also activate expression of the rhaSR operon, in effect RhaS negatively autoregulates this expression by interfering with the ability of the cyclic AMP receptor protein (CRP) to coactivate expression with RhaR to a much greater level. The rhaBAD operon encodes the rhamnose catabolic proteins RhaA (L-rhamnose isomerase), which converts L-rhamnose to L-rhamnulose; RhaB (rhamnulokinase), which phosphorylates L-rhamnulose to form L-rhamnulose-1-P; and RhaD (rhamnulose-1-phosphate aldolase), which converts L-rhamnulose-1-P to L-lactaldehyde and DHAP (dihydroxy acetone phosphate). To maximize the amount of rhamnose in the cell available for induction of expression from a rhamnose-inducible promoter, it is desirable to reduce the amount of rhamnose that is broken down by catalysis, by eliminating or reducing the function of RhaA, or optionally of RhaA and at least one of RhaB and RhaD. E. coli cells can also synthesize L-rhamnose from alpha-D-glucose-1-P through the activities of the proteins RmIA, RmIB, RmIC, and RmID (also called RfbA, RfbB, RfbC, and RfbD, respectively) encoded by the rmIBDACX (or rfbBDACX) operon. To reduce background expression from a rhamnose-inducible promoter, and to enhance the sensitivity of induction of the rhamnose-inducible promoter by exogenously supplied rhamnose, it could be useful to eliminate or reduce the function of one or more of the RmIA, RmIB, RmIC, and RmID.

RmID proteins. L-rhamnose is transported into the cell by RhaT, the rhamnose permease or L-rhamnose: proton symporter. As noted above, the expression of RhaT is activated by the transcriptional regulator RhaS. To make expression of RhaT independent of induction by rhamnose (which induces expression of RhaS), the host cell can be altered so that all functional RhaT coding sequences in the cell are expressed from constitutive promoters. Additionally, the coding sequences for RhaS can be deleted or inactivated, so that no functional RhaS is produced. By eliminating or reducing the function of RhaS in the cell, the level of expression from the rhaSR promoter is increased due to the absence of negative autoregulation by RhaS, and the level of expression of the rhamnose catalytic operon rhaBAD is decreased, further increasing the ability of rhamnose to induce expression from the rha promoter.

Xylose promoter. (As used herein, ‘xylose’ means D-xylose.) The xylose promoter, or ‘xyl promoter’, or PxyiA, means the promoter for the E. coli xylAB operon. The xylose promoter region is similar in organization to other inducible promoters in that the xylAB operon and the xyIFGHR operon are both expressed from adjacent xylose-inducible promoters in opposite directions on the E. coli chromosome (Song and Park, J Bacteriol. 1997 November; 179(22): 7025-7032). The transcriptional regulator of both the PxyiA and PxyiF promoters is XyIR, which activates expression of these promoters in the presence of xylose. The xyIR gene is expressed either as part of the xyIFGHR operon or from its own weak promoter, which is not inducible by xylose, located between the xylH and xyIR protein-coding sequences. D-xylose is catabolized by XyIA (D-xylose isomerase), which converts D-xylose to D-xylulose, which is then phosphorylated by XylB (xylulokinase) to form D-xylulose-5-P. To maximize the amount of xylose in the cell available for induction of expression from a xylose-inducible promoter, it is desirable to reduce the amount of xylose that is broken down by catalysis, by eliminating or reducing the function of at least XylA, or optionally of both XylA and XylB. The xyIFGHR operon encodes XyIF, XyIG, and XyIH, the subunits of an ABC super-family high-affinity D-xylose transporter. The xylE gene, which encodes the E. coli low-affinity xylose-proton symporter, represents a separate operon, the expression of which is also inducible by xylose. To make expression of a xylose transporter independent of induction by xylose, the host cell can be altered so that all functional xylose transporters are expressed from constitutive promoters. For example, the xyIFGHR operon could be altered so that the xyIFGH coding sequences are deleted, leaving XyIR as the only active protein expressed from the xylose-inducible PxyiF promoter, and with the xylE coding sequence expressed from a constitutive promoter rather than its native promoter. As another example, the xyIR coding sequence is expressed from the PxyiA or the promoter in an expression construct, while either the xyIFGHR operon is deleted and xylE is constitutively expressed, or alternatively an xyIFGH operon (lacking the xyIR coding sequence since that is present in an expression construct) is expressed from a constitutive promoter and the xylE coding sequence is deleted or altered so that it does not produce an active protein.

Lactose promoter. The term ‘lactose promoter’ refers to the lactose-inducible promoter for the lacZYA operon, a promoter which is also called lacZpl; this lactose promoter is located at ca. 365603-365568 (minus strand, with the NA polymerase binding (‘−35’) site at ca. 365603-365598, the Pribnow box (‘−10’) at 365579-365573, and a transcription initiation site at 365567) in the genomic sequence of the E. coli K-12 substrain MG1655 (NCBI Reference Sequence NC 000913.2, 1 I-JAN-2012). In some embodiments, inducible coexpression systems of the disclosure can comprise a lactose-inducible promoter such as the lacZYA promoter. In other embodiments, the inducible coexpression systems of the disclosure comprise one or more inducible promoters that are not lactose-inducible promoters.

Alkaline phosphatase promoter. The terms ‘alkaline phosphatase promoter’ and ‘phoA promoter’ refer to the promoter for the phoApsiF operon, a promoter which is induced under conditions of phosphate starvation. The phoA promoter region is located at ca. 401647-401746 (plus strand, with the Pribnow box (‘−10’) at 401695-401701 (Kikuchi et al., Nucleic Acids Res 1981 Nov. 11; 9(21): 5671-5678)) in the genomic sequence of the E. coli K-12 substrain MG1655 (NCBI Reference Sequence NC 000913.3, 16 Dec. 2014). The transcriptional activator for the phoA promoter is PhoB, a transcriptional regulator that, along with the sensor protein PhoR, forms a two-component signal transduction system in E. coli. PhoB and PhoR are transcribed from the phoBR operon, located at ca. 417050-419300 (plus strand, with the PhoB coding sequence at 417,142-417,831 and the PhoR coding sequence at 417,889-419,184) in the genomic sequence of the E. coli K-12 substrain MG1655 (NCBI Reference Sequence NC 000913.3, 16 Dec. 2014). The phoA promoter differs from the inducible promoters described above in that it is induced by the lack of a substance-intracellular phosphate-rather than by the addition of an inducer. For this reason the phoA promoter is generally used to direct transcription of gene products that are to be produced at a stage when the host cells are depleted for phosphate, such as the later stages of fermentation. In some embodiments, inducible coexpression systems of the disclosure can comprise a phoA promoter. In other embodiments, the inducible coexpression systems of the disclosure comprise one or more inducible promoters that are not phoA promoters.

As described herein, it may be advantageous or desirable to remove (e.g., by way of an inducible or constitutive “curing” mechanism) an expression construct described herein, e.g., if the cell line harboring the expression construct is or will be used for commercial purposes. Thus, in some embodiments, the expression construct may comprise a “kill switch.” For example, in embodiment, the expression construct includes a temperature-sensitive origin of replication. Additional curing methods are known in the art and include using detergents and intercalating agents, drugs and antibiotics (Buckner, M. M. C., et al., FEMS Microbiology Reviews, fuy031,42, 2018, 781-804).

### Evaluating the Expressed Library

After generating biomolecules as described herein, including, for example, variant antibodies, the methods of the present disclosure further comprise screening the expressed variants for particular biological characteristics or function as desired.

As used herein, the term “screening” refers to the process in which one or more properties of one or more biomolecules is determined. For example, typical screening processes include those in which one or more properties of one or more members of one or more libraries is/are determined.

Non limiting examples of measurements that can be assayed during the screening of a library include: Activity, Catalytic efficiency (K_{cat}/Kₘ), Catalytic rate constant (k_{cat}), Count/Number, EC50, Enrichment, Epistasis, Fitness, IC50, Inhibition constant (Kᵢ), Maximal rate (Vₘₐₓ), Michaelis constant (Kₘ), Relative activity, Specific activity, Association constant (Kₐ), Binding affinity, Count/Number, Dissociation constant (K_{d}), Equilibrium Constant (K_{D}), ELISA, Energy, Enrichment, Enthalpy of binding (ΔH), Entropy of binding (ΔS), Frequency of occurrence, Gibbs free energy of binding (ΔG), Inhibition constant (Kᵢ), Rate constant of association (Kₒₙ), Rate constant of dissociation (K_{off}), Concentration, Energy, Enrichment, Frequency of occurrence, Minimum inhibitory concentration (MIC), Yield, Antimicrobial resistance, Energy, Enrichment, Frequency of occurrence, Optical density (OD), Bioavailability, EC50, Half-life (t_{1/2}), IC50, Immunogenicity, Toxicity, Concentration, Energy, Fractional increase in solubility, Insoluble fraction, Oligomerization state, Soluble fraction, Energy, Frequency of occurrence, Relative activity, Relative affinity, Relative k_{cat}, Relative k_{cat}/kₘ, Relative K_{d}, Brightness, Emission wavelength (λₑₘ), Energy, Excitation wavelength (λex), Extinction coefficient, Fluorescence intensity, Maturation half-time, Photobleaching half-time, pKa, Quantum yield, Constant pressure heat capacity of unfolding (ΔCₚ), Count/Number, Denaturant concentration at midpoint of unfolding transition (Cₘ), Energy, Enthalpy of unfolding (ΔH), Entropy of unfolding (ΔS), Equilibrium constant (K), Gibbs free energy of folding/unfolding (ΔG), Melting temperature (Tₘ), Rate of folding (k_{F}), Rate of unfolding (kᵤ), Slope of chevron plot (m), Slope of the denaturant unfolding curve/cooperativity value (m), Temperature of maximum stability, Thermal tolerance, β-Tanford value, viscosity, and φ-value. In some embodiments, the protein identifier is a name or a full length protein sequence.

In an embodiment, the screening method of the present disclosure measures binding affinities.

In further embodiments, the screening method measures expression levels.

Periplasmic and cytoplasmic expression. In certain aspects of the disclosure, the antibody or antibody fragments of the present disclosure are expressed in the periplasmic space membrane or cytoplasm of a host bacterial cell.

The periplasmic compartment is contained between the inner and outer membranes of Gram negative cells (see, e.g., Oliver, 1996). As a subcellular compartment, it is subject to variations in size, shape and content that accompany the growth and division of the cell. Within a framework of peptidoglycan heteroploymer is a dense milieu of periplasmic proteins and little water, lending a gel-like consistency to the compartment (Hobot et al., 1984; van Wielink and Duine, 1990). The peptidoglycan is polymerized to different extents depending on the proximity to the outer membrane, close-up it forms the murein sacculus that affords cell shape and resistance to osmotic lysis.

The outer membrane (see Nikaido, 1996) is composed of phospholipids, porin proteins and, extending into the medium, lipopolysaccharide (LPS). The molecular basis of outer membrane integrity resides with LPS ability to bind divalent cations (Mg2+ and Ca2+) and link each other electrostatically to form a highly ordered quasi-crystalline ordered “tiled roof” on the surface (Labischinski et al., 1985). The membrane forms a very strict permeability barrier allowing passage of molecules no greater than around 650 Da (Burman et al., 1972; Decad and Nikaido, 1976) via the porins. The large water filled porin channels are primarily responsible for allowing free passage of mono and disaccharides, ions and amino acids into the periplasm compartment (Nikaido and Nakae, 1979; Nikaido and Vaara, 1985).

To detect antibodies and antibody fragments in the periplasmic space or cytoplasm requires specific labeling with appropriate fluorescent ligands. However, the permeability barrier of the outer membrane prevents the diffusion of labeled ligands into the periplasm and cytoplasm to access the expressed antibody or antibody fragment. Such diffusion can be aided by permeabilizing the outer membrane of the host cell as described below.

An antibody or antibody fragment that is expressed in the periplasm could be tethered to the inner membrane of a Gram negative bacteria by means of a short lipoprotein signal or an engineered lipoprotein. The binding between the antibody or antibody fragment and the labeled ligand will prevent diffusing out of a bacterial cell. In this way, molecules of the labeled ligand can be retained in the periplasm of the bacterium comprising a permeabilized outer membrane. Alternatively, the periplasm can be removed and the generated spheroplasts incubated with the labeled ligand, whereby the Fc domain will cause retention of the bound candidate molecule since Fc domains are shown to associate with the inner membrane.

For antibody or antibody fragments that are expressed and retained in the host cell cytoplasm, the labeling procedure can include fixation, so that the expressed polypeptide of interest remains associated with its host cell.

Permeabilization of the outer membrane. In one embodiment of the disclosure, methods are employed for increasing the permeability of the outer membrane to one or more labeled ligands. This can allow screening access of labeled ligands otherwise unable to cross the outer membrane. However, certain classes of molecules, for example, hydrophobic antibiotics larger than the 650 Da exclusion limit, can diffuse through the bacterial outer membrane itself, independent of membrane porins (Farmer et al., 1999). The process may actually permeabilize the membrane on doing so (Jouenne and Junter, 1990). Such a mechanism has been adopted to selectively label the periplasmic loops of a cytoplasmic membrane protein in vivo with a polymyxin B nonapeptide (Wada et al., 1999). Also, certain long chain phosphate polymers (100 Pi) appear to bypass the normal molecular sieving activity of the outer membrane altogether (Rao and Torriani, 1988).

Conditions have been identified that lead to the permeation of ligands into the periplasm without loss of viability or release of the expressed proteins from the cells, but the disclosure may be carried out without maintenance of the outer membrane. As demonstrated herein Fc domains expressed or anchored candidate binding polypeptides in the periplasmic space the need for maintenance of the outer membrane (as a barrier to prevent the leakage of the binding protein from the cell) to detect bound labeled ligand is removed. As a result, cells expressing binding proteins anchored to the outer (periplasmic) face of the cytoplasmic membrane can be fluorescently labeled simply by incubating with a solution of fluorescently labeled ligand in cells that either have a partially permeabilized membrane or a nearly completely removed outer membrane.

The permeability of the outer membrane of different strains of bacterial hosts can vary widely. It has been shown previously that increased permeability due to OmpF overexpression was caused by the absence of a histone like protein resulting in a decrease in the amount of a negative regulatory mRNA for OmpF translation (Painbeni et al., 1997). Also, DNA replication and chromosomal segregation is known to rely on intimate contact of the replisome with the inner membrane, which itself contacts the outer membrane at numerous points. In one embodiment, a host for library screening applications is E. coli ABLEC strain, which additionally has mutations that reduce plasmid copy number. As described herein, in another embodiment the E. coli SoluPro stain is a suitable host for library screening applications (SoluPro™ E. coli (See, e.g., WO/2014/025663 and WO/2017/106583)).

Treatments such as hyperosmotic shock can improve labeling significantly. It is known that many agents including calcium ions (Bukau et al., 1985) and even Tris buffer (Irvin et al., 1981) alter the permeability of the outer-membrane. Further, phage infection stimulates the labeling process. Both the filamentous phage inner membrane protein pIII and the large multimeric outer membrane protein pIV can alter membrane permeability (Boeke et al., 1982) with mutants in pIV known to improve access to maltodextrins normally excluded (Marciano et al., 1999). Using the techniques of the disclosure, comprising a judicious combination of strain, salt and phage, a high degree of permeability may be achieved (Daugherty et al., 1999). Cells comprising anchored or periplasm-associated polypeptides bound to fluorescently labeled ligands can then be easily isolated from cells that express binding proteins without affinity for the labeled ligand using flow cytometry or other related techniques. However, in some cases, it will be desired to use less disruptive techniques in order to maintain the viability of cells. EDTA and Lysozyme treatments may also be useful in this regard.

Fixation. In one embodiment of the disclosure, methods are employed for retaining antibody or antibody fragment within host cells by fixing the host cells with a crosslinking reagent, such as one or more aldehydes (paraformaldehyde, glutaraldehyde, formaldehyde), applied in solution. Fixation of antibody or antibody fragments within the host cells using one or more aldehydes is an example of electrophile/nucleophile chemistry, where the aldehydes are the electrophiles and the antibody or antibody fragment supplies the nucleophilic centers, such as the amine groups in polypeptides and the N7-position of guanine residues of polynucleotides. Crosslinking reagents are typically bifunctional and can react with the antibody or antibody fragment at one end, and with a component of the host cell (DNA, RNA, cytoskeleton, membrane, cell wall, or protein complexed to one of these components) at the other end. Many different types of crosslinking reagents are commercially available (ThermoFisher Scientific Inc., Waltham, Massachusetts). Another method of retaining the antibody or antibody fragment within the host cell involves including a polynucleotide sequence encoding a polypeptide or polynucleotide that associates with a structure of the host cell, such as a cytoskeletal component or other cytoplasmic structure, within the coding sequence for the gene product of interest. For example, particularly in prokaryotic host cells, attaching all or part of the cytoskeletal MreB protein or its analog to a gene product of interest can cause the antibody or antibody fragments to become associated with the inner cell membrane through the interaction of MreB with MreC or an analogous protein.

Labeling the Nucleic Acids of Host Cells. The DNA and other nucleic acids of live host cells can be labeled with dyes that are uncharged (such as Hoechst 33342) or that contain conjugated systems to distribute any charge, making them able to permeate cells. However, a live host cell may transport dye back out of the cell. Host cells can be fixed and/or permeabilized to allow DNA-labeling compound(s) to enter and remain in the host cells. Compounds that label DNA in fixed cells include propidium iodide (PI), 7-aminoactinomycin-D (7-AAD), and 4′6′-diamidino-2-phenylindole (DAPI). Thus, in some examples, a DNA stain is utilized to identify live cells in the population.

### Labeled Target Ligands

Detection of an antibody or antibody fragment that is expressed in a host cell involves the association of the antibody or antibody fragment with a ligand that is labeled with a detectable agent such that a detectable signal is associated with that particular host cell.

Three separate ligands could be used, individually or in any combination, to detect an antibody or antibody fragment: antigen to specifically bind the antigen-binding domain, an anti-Fc antibody to specifically bind properly folded and/or assembled Fc region, and an anti-light-chain antibody to specifically bind properly folded and/or assembled light chain.

Ligands can be labeled for example, by linking the ligand to at least one detectable agent to form a conjugate. For example, it is conventional to link or covalently bind or complex at least one detectable molecule or moiety. A “label” or “detectable label” is a compound and/or element that can be detected due to specific functional properties, and/or chemical characteristics, the use of which allows the ligand to which it is attached to be detected, and/or further quantified if desired. Examples of labels that could be used include, but are not limited to, enzymes, radiolabels, haptens, fluorescent labels, phosphorescent molecules, chemiluminescent molecules, chromophores, luminescent molecules, photoaffinity molecules, colored particles, or ligands, such as biotin.

In one embodiment of the disclosure, a visually-detectable marker is used such that automated screening of cells for the label can be carried out. Examples of agents that may be detected by visualization with an appropriate instrument are known in the art, as are methods for their attachment to a desired ligand (see, e.g., U.S. Pat. Nos. 5,021,236; 4,938,948; and 4,472,509, each incorporated herein by reference). Such agents can include paramagnetic ions; radioactive isotopes; fluorochromes; NMR-detectable substances; and substances for X-ray imaging. In particular, fluorescent labels are beneficial in that they allow use of flow cytometry for isolation of cells expressing a desired binding protein or antibody.

In certain embodiments, the fluorochrome is selected from the group consisting of PerCP; R-PE; DyLight-488; Alexafluor 488; Alexafluor 633; APC; PE; DyLight-633; 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; Bodipy 492/515; Bodipy 493/503; Bodipy 500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; Brilliant Sulphoflavin FF; BTC; BTC-5N; Calcein; Calcein Blue; Calcium Crimson; Calcium Green; Calcium Green-1Ca2+ Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM | Methylcoumarin; CTC; CTC Formazan; Cy2®; Cy3.1 8®; Cy3.5®; Cy3®; Cy5.1 8®; Cy5.5®; Cy5®; Cy7®; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; Fura Red® (high pH); Fura Red®/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green® 488; Oregon Green® 500; Oregon Green@ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP® (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Reds; Texas Red-X™ conjugate; Thiadicarbocyanine (DiSC3); Thiazine Red™; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelso ThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophores (which can be activated with light or other electromagnetic energy source), or a combination thereof.

Another type of ligand conjugate is where the ligand is linked to a secondary binding molecule and/or to an enzyme (an enzyme tag) that will generate a colored product upon contact with a chromogenic substrate. Examples of such enzymes include urease, alkaline phosphatase, (horseradish) hydrogen peroxidase, or glucose oxidase. In such instances, it will be desired that cells selected remain viable. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149; and 4,366,241, each incorporated herein by reference.

Molecules containing azido groups may be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide-binding proteins in crude cell extracts (Owens and Haley, 1987; Atherton et al., 1985). The 2- and 8-azido nucleotides have also been used to map nucleotide-binding domains of purified proteins (Khatoon et al., 1989; King et al., 1989; Dholakia et al., 1989) and may be used as ligand binding agents.

Labeling can be carried out by any of the techniques well known to those of skill in the art. For instance, FcR polypeptides can be labeled by contacting the ligand with the desired label and a chemical oxidizing agent, such as sodium hypochlorite, or an enzymatic oxidizing agent, such as lactoperoxidase. Similarly, a ligand exchange process could be used. Alternatively, direct labeling techniques may be used, e.g., by incubating the label, a reducing agent such as SNCl2, a buffer solution such as sodium-potassium phthalate solution, and the ligand. Intermediary functional groups on the ligand could also be used, for example, to bind labels to a ligand in the presence of diethylenetriaminepentaacetic acid (DTPA) or ethylenediaminetetraacetic acid (EDTA).

Other methods are also known in the art for the attachment or conjugation of a ligand to its conjugate moiety. Some attachment methods involve the use of an organic chelating agent, such as diethylenetriaminepentaacetic acid anhydride (DTPA); ethylenediaminetetraacetic acid; N-chloro-p-toluenesulfonamide; and/or tetrachloro-3α-6α-diphenyl glycoluril-3 attached to the ligand (U.S. Pat. Nos. 4,472,509 and 4,938,948, each incorporated herein by reference). FcR polypeptides also may be reacted with an enzyme in the presence of a coupling agent such as glutaraldehyde or periodate. Conjugates with fluorescein markers can be prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzamide or N-succinimidyl-3-(4-hydroxyphenyl) propionate. In still further aspects an FcR polypeptide may be fused to a reporter protein, such as an enzyme as described supra or a fluorescence protein.

Automated Screening with FACS

In another aspect, the present disclosure provides a method of sorting the host cell population based on the specific binding of the expressed biomolecule—e.g., an antibody or antibody fragment to a target antigen, comprising providing a diverse library of transformed host cells expressing a diverse library of biomolecules (e.g., binding proteins) as disclosed herein; contacting the host cells with the target antigen; and sorting host cells based on their binding to the target antigen, thereby identifying subpopulations of cells that specifically bind to a target antigen.

In another aspect, the disclosure provides a method of sorting the host cell population based on the specific binding of the expressed antibody or antibody fragment to a first target antigen probe and a second non-antigen probe simultaneously, the method comprising: providing a diverse library of transformed host cells expressing a diverse library of binding proteins disclosed herein; contacting the host cells with the first and second probes; and sorting host cells based on their binding to the first and second probes, thereby identifying subpopulations of cells that specifically bind to a first and a second probe simultaneously.

In certain embodiments of the methods disclosed herein, host cells that bind to the first and/or second probe are selected by Magnetic Activated Cell Sorting (MACS) using magnetically labeled antigen.

In certain embodiments of the methods disclosed herein, host cells that bind to the first and/or second probe are selected by Fluorescence Activated Cell Sorting (FACS) using fluorescently labeled antigen.

FACS is a powerful tool that allows analysis of multiple individual cell parameters, providing the ability to separate a heterogeneous suspension of cells into a homogenous fraction of single cells based on fluorescence and light scattering properties. Instruments for carrying out flow cytometry are known to those of skill in the art and are commercially available to the public. Examples of such instruments include, but are not limited to, BD FACSAria™-Ilu instrument (Becton Dickinson), COULTER EPICS XL/XL-MCL (Coulter Epics Division), and MoFlo XDP (Beckman Coulter), Attune N×T Flow Cytometer (ThermoFisher). Once cells are sorted, gates or boundaries are placed around populations of cells with common characteristics, usually forward scatter (FSC), side scatter (SSC) and the fluorescence of the labels detecting expressed proteins or labeled DNA. FSC and SSC give an idea of the size and granularity of the cells respectively. By setting specific gates, the subpopulations of host cells can be separated and collected into a plurality of collection tubes for investigation and/or quantification of the subpopulations of interest. In some embodiments of the methods disclosed herein, host cells are gated according to antigen binding affinity and expression levels of the expressed antibodies or antibody fragments. In particular examples, the gating parameters also identify and exclude aggregated cells or non-cellular debris, in order to measure signal substantially only from single cells. This reduces artifacts of increased expression of the product of interest due to cell “clumping” rather than actual increase due to the particular genetic diversity of a cell.

In certain embodiments, the methods disclosed herein optionally comprise the rescreening of sorted host cell subpopulations from the plurality of collection tubes sorted by FACS to validate the calculated Kps an additional technique. As used herein, the term “optional” or “optionally” means that the subsequent described event, circumstance or substituent may or may not occur, and that the description includes instances where the event or circumstance occurs and instances where it does not.

Suitable alternative methods for rescreening and measuring binding affinities are known in the art and can be selected from the group consisting of ELISA, Surface Plasmon Resonance (SPR), Biolayer Interferometry and flow cytometry derived binding curves.

In one embodiment, the rescreening is performed by SPR. A BIAcore-2000 or BIAcore-3000 real-time kinetic interaction analysis system (Biacore Inc., Piscataway, N.J.) may then be used to determine association (kₒₙ) and dissociation (k_{off}) constants (Karlsson, R., Michaelsson, A. & Mattsson, L., J Immunol Methods 145(1-2): 229-40 (1991)) of the antibody fragments in binding interactions with immobilized antigen, according the manufacturer's instructions. The K_{D} may be calculated from k_{off}/kₒₙ, as known in the art.

In some embodiments, the binding affinities of the antibodies described herein are measured by array surface plasmon resonance (SPR), according to standard techniques (Abdiche, et al. (2016) MAbs 8:264-277). Briefly, antibodies were immobilized on a HC 30M chip at four different densities/antibody concentrations. Varying concentrations (0-500 nM) of antibody targets are then bound to the captured antibodies. Kinetic analysis is performed using Carterra software to extract association and dissociation rate constants (kₐ and k_{d}, respectively) for each antibody. Apparent affinity constants (K_{D}) are calculated from the ratio of k_{d}/kₐ. In some embodiments, the Carterra LSA Platform is used to determine kinetics and affinity. In other embodiments, binding affinity can be measured, e.g., by surface plasmon resonance (e.g., BIAcore™) using, for example, the IBIS MX96 SPR system from IBIS Technologies or the Carterra LSA SPR platform, or by Bio-Layer Interferometry, for example using the Octet™ system from ForteBio. In some embodiments, a biosensor instrument such as Octet RED384, ProteOn XPR36, IBIS MX96 and Biacore T100 is used (Yang, D., et al., J. Vis. Exp., 2017, 122:55659).

K_{D} is the equilibrium dissociation constant, a ratio of k_{off}/kₒₙ, between the antibody and its antigen. K_{D} and affinity are inversely related. The K_{D} value relates to the concentration of antibody and so the lower the K_{D} value (lower concentration) and thus the higher the affinity of the antibody. Antibody, including reference antibody and variant antibody, K_{D} according to various embodiments of the present disclosure can be, for example, in the micromolar range (10⁻⁴ to 10⁻⁶), the nanomolar range (10⁻⁷ to 10⁻⁹), the picomolar range (10⁻¹⁰ to 10⁻¹²) or the femtomolar range (10⁻¹³ to 10⁻¹⁵). In some embodiments, antibody affinity of a variant antibody is improved, relative to a reference antibody, by approximately 5, 10, 15, 20, 25, 30, 35, 40, 45, or 50% or more. The improvement may also be expressed relative to a fold change (e.g., 2×, 4×, 6×, or 2-, 3-, 4-, 5-, 6-, 7-, 8-, 9-, 10-fold or more improvement in binding activity, etc.) and/or an order of magnitude (e.g., 10⁷, 10⁸, 10⁹, etc.).

The present disclosure also provides methods which produce reliable counts of each sequence variant. When qaACE is performed with the objective of enriching for high-affinity variants, all that matters is retrieving such variants from a high-fluorescence gate at least once. It is not necessary to retrieve the same sequence several times. Even when it is—for example to enhance confidence—counting the same sequence variants just a handful of times may be sufficient. Conversely, when the objective is to accurately determine a fluorescence score (a surrogate of affinity) of each sequence variant, it is imperative to retrieve several tens if not hundreds of reads for each sequence variant. Because of technical noise, not all reads of the same variant will originate from the same gate. Thus, one can compute the fluorescence score of a variant by averaging the midpoint fluorescence intensity of each gate weighted by the number of reads of that variant originating from each gate. Alternatively, various scores can be calculated using the read count of an individual in the library and the fluorescence of each gate the individual was observed in. Score types include a “slope” fit of a linear model across the distribution and/or an estimation of the true fluorescence distribution of the individual. Such fluorescence scores can be computed accurately only if a sufficient number of independent reads is acquired. This is true for any count-based application of NGS, such as RNA-seq. To ensure that enough reads of the same variant can be observed, the library size must be restricted. Otherwise, if the library size is greater than sequencing coverage, each variant can be observed only from 0 to a handful of reads, which are not sufficient to compute an accurate fluorescence score. The tradeoff between library size and assay quantitativeness is well understood in the literature describing, for example, Deep Mutational Scanning.

### Next Generation Sequencing of Sorted Host Cell Subpopulations

In some embodiments, the subpopulations of host cells sorted into a plurality of collection tubes (i.e., “bins”) are further characterized to gain insight into possible mutational correlations or relationships that lead to a desired functional change. In some embodiments, further characterizing these subpopulations comprises analyzing variants individually through sequencing, to identify the specific mutation or mutations that are connected to the change in characteristic (such as a highly functional characteristic). Individual mutant variants of the biomolecule can be isolated through standard molecular biology techniques for later analysis of function.

The term “sequence” is used herein to refer to the order and identity of any biological sequences including but not limited to a whole genome, whole chromosome, chromosome segment, collection of gene sequences for interacting genes, gene, nucleic acid sequence, protein, peptide, polypeptide, polysaccharide, etc. In some contexts, a “sequence” refers to the order and identity of amino acid residues in a protein (i.e., a protein sequence or protein character string) or to the order and identity of nucleotides in a nucleic acid (i.e., a nucleic acid sequence or nucleic acid character string). A sequence may be represented by a character string. A “nucleic acid sequence” refers to the order and identity of the nucleotides comprising a nucleic acid. A “protein sequence” refers to the order and identity of the amino acids comprising a protein or peptide. “Codon” refers to a specific sequence of three consecutive nucleotides that is part of the genetic code and that specifies a particular amino acid in a protein or starts or stops protein synthesis.

In some embodiments, further characterizing the host subpopulations comprises high throughput sequencing or next generation sequencing (NGS) of the plurality of host sub-populations comprising high binders, low binders and everything in between. This approach may, in some embodiments, may allow for the rapid identification of mutations that are overrepresented in the one or more sub-populations.

As used herein, the terms “next generation sequencing (NGS)” and “high-throughput sequencing” are sequencing techniques that parallelize the sequencing process, producing thousands or millions of sequences at once. Examples of suitable next-generation sequencing methods include, but are not limited to, single molecule real-time sequencing (e.g., Pacific Biosciences, Menlo Park, California), ion semiconductor sequencing (e.g., Ion Torrent, South San Francisco, California), pyrosequencing (e.g., 454, Branford, Connecticut), sequencing by ligation (e.g., SOLID sequencing of Life Technologies, Carlsbad, California), sequencing by synthesis and reversible terminator (e.g., Illumina, San Diego, California), nucleic acid imaging technologies such as transmission electron microscopy, and the like.

NGS can produce high throughput data indicating the functional effect of the library members. In embodiments wherein one or more libraries represents every possible mutation of every monomer location, such high throughput sequencing can evaluate the functional effect of every possible mutation. Such sequencing can also be used to evaluate one or more highly or less functional sub-populations of a given library, which in some embodiments may lead to identification of mutations that result in improved and decreased function respectively.

In certain embodiments, the methods disclosed herein may comprise amplification of DNA obtained from the sorted host cell subpopulations. In some embodiments, RNA can also be recovered from selected host cells, reverse-transcribed into DNA. DNA amplification is useful when the quantity of isolated DNA is inadequate for NGS. If the cells that were FACS sorted comprise cells that express the library of antibody or antibody fragment variants from a plasmid (for example, E. coli cells transformed with a plasmid expression vector), these plasmids can be isolated, for example through a miniprep. Conversely if the library of biomolecule variants has been integrated into the genomes of the FACs sorted cells, this DNA region can be PCR amplified and, optionally, subcloned into a suitable vector for further characterization using methods known in the art. Thus, the end product of library screening is a DNA library representing the initial, or ‘naive’, library, as well as one or more DNA libraries containing sub-populations of the naive library which comprise highly functional mutant variants of the biomolecule identified by the screening processes described herein.

An Example of one embodiment of the sRCA amplification technique is provided below.

In an embodiment, the DNA amplification step disclosed herein further comprises the addition of barcodes or Unique Molecular Indices (UMI) to the DNA isolated from the sorted host cell subpopulations.

As used herein, the term “barcode” refers to a nucleic acid sequence that is used to identify a single cell or a subpopulation of cells. Barcode sequences can be linked to a target nucleic acid of interest during amplification and used to trace back the amplicon to the cell from which the target nucleic acid originated. A barcode sequence can be added to a target nucleic acid of interest during amplification by carrying out PCR with a primer that contains a region comprising the barcode sequence and a region that is complementary to the target nucleic acid such that the barcode sequence is incorporated into the final amplified target nucleic acid product (i.e., amplicon). Barcodes can be included in either the forward primer or the reverse primer or both primers used in PCR to amplify a target nucleic acid. A barcode can be any number of nucleotides in length. A barcode can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides in length. In some cases, the barcode is more than 30 nucleotides in length. A barcode can be generated by degenerate oligonucleotide synthesis. A barcode can be rationally designed or user-specified.

As used herein, the term “Unique Molecular Indices (UMI)” refers to randomized nucleotides sequences applied to or identified in DNA molecules that may be used to distinguish individual DNA molecules from one another. Since UMIs are used to identify DNA molecules, they are also referred to as unique molecular identifiers. See, e.g., Kivioja, Nature Methods 9, 72-74 (2012). UMIs may be sequenced along with the DNA molecules with which they are associated to determine whether the read sequences are those of one source DNA molecule or another. The term “UMI” is used herein to refer to both the sequence information of a polynucleotide and the physical polynucleotide per se.

The addition of UMIs (random molecular barcodes) to amplicons during the first few PCR cycles will allow to uniquely tag each template molecule. Down the line, when sequencing will yield identical reads, one will be able to disambiguate sequencing/PCR duplicates (not of interest, to be counted only once) from identical but molecularly independent templates (biologically interesting, each to be counted). UMIs are widespread in several modern molecular biology protocols leveraging PCR with downstream NGS endpoints.

The amplification reaction according to the present method may be either a non-isothermal method or an isothermal method.

Suitable methods for non-isothermal amplification include polymerase chain reaction (PCR), (Saiki et al. Science (1985) 230:1350-1354), and ligase chain reaction (LCR) (Landegren et al. Science (1988) 241:1077-1080).

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al, editors, PCR: A Practical Approach and PCR2: A Practical Approach (IRL Press, Oxford, 1991 and 1995, respectively).

Suitable isothermal amplification methods may be selected from the group of helicase-dependent amplification (HDA) (Vincent et al. EMBO Rep (2004) 5 (8): 795-800), thermostable HDA (tHDA) (An et al. J. Biol. Chem. (2005) 280 (32): 28952-28958), strand displacement amplification (SDA) (Walker et al. Nucleic Acids Res. (1992) 20 (7): 1691-1696), multiple displacement amplification (MDA) (Dean et al. Proc. Natl. Acad. Sci. USA (2002) 99 (8): 5261-5266), selective rolling-circle amplification (sRCA, as described herein), restriction aided RCA (Wang et al. Genome Res (2004) 14:2357-2366), single primer isothermal amplification (SPIA) (Dafforn et al. Biotechniques (2004), 37 (5): 854-857), transcription mediated amplification (TMA) (Vuorinen et al. J. Clin. Microbiol. (1995) 33:1856-1859), nicking enzyme amplification reaction (NEAR) (Maples et al. US2009017453), exponential amplification reaction (EXPAR) (Van Ness et al. Proc. Natl. Acad. Sci. USA (2003) 100 (8): 4504-4509), loop mediated isothermal amplification (LAMP) (Notomi et al. Nucleic Acids Res. (2000) 28 (12): e63), recombinase polymerase amplification (RPA) (Piepenburg et al. PloS Biol. (2006) 4 (7): 1115-1120), nucleic acid sequence based amplification (NASBA) (Kievits et al. J. Virol. Methods (1991) 35:273-286), smart-amplification process (SMAP) (Mitani et al. Nat. Methods (2007) 4 (3): 257-262).

In an embodiment, the amplification method is the selective rolling-circle amplification (sRCA) method.

As used herein, the term “rolling circle amplification (RCA)” refers to an isothermal acid amplification reaction that amplifies a circular nucleic acid template (e.g., single/double stranded DNA circles) using a strand-displacing polymerase. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers comprising tandem repeat units of the circular nucleic acid template sequence. The rolling circle amplification may be a linear RCA (LRCA), exhibiting linear amplification kinetics (e.g., RCA using a single, specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential amplification kinetics featuring a cascade in series of multiple-hybridization, primer-extension, and strand-displacement events involving both the primers and both strands. This often generates a discrete set of concatemeric, double-stranded nucleic acid amplification products. The RCA may be performed in vitro under isothermal conditions using a suitable nucleic acid polymerase such as Phi29 DNA polymerase. Suitable polymerases possess strand displacement DNA synthesis ability. In some embodiments, the Phi29 DNA polymerase possesses a 70,000 base pair strand displacement capability that allows primers to bind in a relatively small portion of the template, while still effectively amplifying the entire sequence. In an embodiment, the rolling circle amplification employs primers designed to target conserved regions of antibiotic markers and their flanking regions in the template (selective RCA or sRCA). In further embodiments, the template is plasmid DNA. The sRCA primer design allows for the amplification of a plasmid carrying a specific resistance marker in cells containing plasmids carrying multiple other resistance markers, while avoiding off-target amplification of other plasmids or genomic DNA. In additional embodiments, sRCA primers may also be used in combination to amplify two or more plasmids from the same cell.

### Enrichment Scores

The methods disclosed herein further comprise the calculation of enrichment scores (including, for example, qaACE affinity scores or binding scores) from the identities of the individual antibody or antibody fragment variant sequences observed across the affinity gates and the Kd measurements associated with each sequence including strong binders and weak binders, thereby correlating sequence to a functional property.

The enrichment scores generated by the methods disclosed herein make up a dataset for training a supervised machine learning model to learn the relationship between sequence and function (i.e., binding).

As used herein the term “training data” refers to data items that are examples of one or more categories to be learned, each exampleeither belonging or not belonging to the one or more categories. Categories refer to classes, divisions or partitions of the training data regarded as having a particular shared characteristic. In other words, training data refers to data items provided as examples or counterexamples of a property that the machine should learn. Training data is the most common input of machine learning methods.

The enrichment scores generated by the qaACE assay are an ideal data type for training a machine learning model because of the accuracy and high throughput.

In one embodiment the enrichment scores (for example, qaACE affinity scores) can be calculated as described in the example below. Briefly, the raw read sequences from the high-through sequencing are preprocessed and quality controlled before mapping to the reference sequence.

As used herein, the term “reference biomolecule” refers to a biomolecule, which is generally, although not necessarily, to which a target biomolecule is compared. Thus, for example, a reference sequence is a sequence to which a target sequence is compared, in order to identify potential or actual sequence variations in the target sequence, relative to the reference sequence.

In NGS, low data quality may be generated from several sources including, but not limited to, adapter contamination, base content biases, overrepresented sequences, and errors in library preparation or sequencing steps. Quality control (QC) and preprocessing are effective ways to eliminate possible sequencing errors. Preprocessing and QC steps include, for example, adapter trimming, base correction, overlapping analysis, polyG tail trimming, sliding window cutting, global trimming and quality filtering. QC and preprocessing of sequencing genera for clean data to be produced for subsequent bioinformatic analysis for example alignment to the reference sequence.

In an embodiment, the count of the “clean” sequences are then normalized within each gate (i.e., within each sorted subpopulation from which a specific molecule was sequenced from), by dividing it with the total number of reads from that gate/subpopulation and multiplying that result by 1 million.

In a further embodiment, a binding score (qaACE score) is then assigned to each unique DNA sequence by taking a weighted average of the normalized counts across the sorting gates. In some embodiments, the weights are assigned linearly, whereby the gate with the lowest signal getting the weight of 1, and the gate with the highest signal getting the weight equal to the total number of gates used in the experiment. In an embodiment, the multiple measurements obtained for each amino-acid sequence in the library, for example from multiple synonymous DNA variants and multiple replicate FACS sorts, are aggregated into a single data-point using their mean value. In some embodiments, the multiple measurements can be used as additional QC step to check for inconsistencies across the replicates. In further embodiments, noisy data can be discarded by getting rid of sequences where the standard deviation across the measurements is above a manually derived threshold of 1.

### Training a Machine Learning Model

The methods described herein comprise generating training data to train a machine learning model to predict sequence-property characteristics.

As used herein, the term “machine learning” may refer to algorithms that give a computer the ability to autonomously (i.e., without being explicitly programmed) learn and improve from an experience (e.g., training data), thereby allowing them to extract patterns from data and make predictions. Thus, trained machine learning models can accurately analyze data with unknown outcomes, based on lessons learned from training data.

As used herein, the term “machine learning model” may refer to a computer representation that can be tuned (e.g., trained) based on inputs to approximate unknown functions. In particular, the term “machine-learning model” can include a model that utilizes algorithms to learn from, and make predictions on, known data by analyzing the known data to learn to generate outputs that reflect patterns and attributes of the known data. Example machine learning models can include, but are not limited to: decision trees, support vector machines, artificial neural networks, Bayesian networks, perceptron (“P”), feed forward (“FF”), radial basis network (“RBF”), deep feed forward (“DFF”), recurrent neural network (“RNN”), random forest learning, long/short term memory (“LSTM”), gated recurrent unit (“GRU”), auto encoder (“AE”), variational AE (“VAE”), denoising AE (“DAE”), sparse AE (“SAE”), markov chain (“MC”), Hopfield network (“HN”), Boltzmann machine (“BM”), deep belief network (“DBN”), deep convolutional network (“DCN”), deconvolutional network (“DN”), deep convolutional inverse graphics network (“DCIGN”), generative adversarial network (“GAN”), liquid state machine (“LSM”), extreme learning machine (“ELM”), echo state network (“ESN”), deep residual network (“DRN”), kohonen network (“KN”), support vector machine (“SVM”), neural turing machine (“NTM”), a combination thereof, and/or the like.

In an embodiment, a convolutional neural network may be trained to predict the relative binding affinity of unseen antibody or antibody fragment sequences for a target.

As used herein, the term “training data” can refer to data and/or data sets used to train one or more machine learning models. In the present disclosure, the training data comprise the enrichment scores calculated from the sequencing and the functional data from the binding affinity measurements. In some cases, multiple types of functional data (e.g., rate constant data and thermal stability data) are provided together in the training data. Training data can be subdivided into several different datasets for example; 1. A “training (or model-building) set” refers to a subset of the training data that one or more models are fitted to (trained) and built upon. 2. A “validation (or prediction) set” refers to a subset of the training data held back from training the model and this is used to test the predictive power or performance of the trained model. This is called cross validation. Therefore, the term “cross validation” refers to the use of one set of data, to test the generalizability of the ability of a model trained on a different set of data, to predict the value of the dependent variable. The phrase “predictive power” refers to the ability of a model to correctly predict (i.e., the ability to correctly anticipate unseen data) the values of a dependent variable. For example, in the present disclosure, the predictive power of the model to be trained refers to its ability to predict binding affinities from sequence information.

In one embodiment, the machine learning model may be validated using holdout data that has labeled actual outcomes. Validation may include applying the machine learning model to the holdout data to generate a predicted output that may be compared to the labeled actual outcomes. The machine learning model may then be based on the comparison using sufficiency criteria. The sufficiency criteria applied may vary depending upon the size of the training data set available for training, the performance of previous iterations of models, or user-specified performance requirements. If the machine learning model does not meet sufficiency criteria, the machine learning model may be adjusted in one or more manners. For example, one or more weights of the machine learning model may be adjusted, the machine learning model may have training on additional training data, a different architecture or type of machine learning model may be selected, or some other suitable change to the machine learning model.

In some embodiments, the machine learning model may be trained using a supervised machine-learning program or algorithm meaning it is trained using labeled or classified data. For example, the enrichment score training data of the present disclosure constitute labeled data. In some embodiments, the machine learning model may be trained using an unsupervised machine-learning program or algorithm meaning it is trained using unlabeled and unclassified data. The training data may be unlabeled, or the training data set may be labeled, such as by a human. In some embodiments, the machine-learning program or algorithm may employ a combined learning module or program that learns in two or more features or feature datasets in a particular area of interest.

Machine-learning may involve identifying and recognizing patterns in existing data in order to facilitate making predictions for subsequent data. In some embodiments, due to the processing power requirements of training machine learning models, the selected model may be trained using additional computing resources (e.g., cloud computing resources) based upon data provided by a server.

Once such a model is generated, antibody sequences that are designed to improve binding to a target can be predicted and tested. Data from additional experiments may be used to improve the model's ability to accurately predict outcomes. Such models may design previously unseen sequences with both highly uncertain and a range of predicted affinities. These designs can be tested using the same host cell display, and the observed high-throughput affinity data can be used to improve the models to enable the prediction of high-affinity and highly-specific binders. The recent commercialization of array-based oligonucleotide synthesis allows for a million specified DNA sequences to be manufactured at modest cost. The predicted antibody sequences can be synthesized with a range of predicted affinities by our models for a given target using these oligonucleotide services. These sequences can be expressed on high-throughput display platforms, and then affinity experiments followed by sequencing can be performed to determine the accuracy of the models of antibody affinity. The resulting affinity data may be used to further train machine learning models to enable the prediction of highly target-specific antibodies.

While various techniques of using a machine learning model to predict sequence-property characteristics are described herein, it is worth noting that in some embodiments a statistical model may be used in addition or in alternative to a machine learning model. The statistical model may include a statistical model that may be parametric, nonparametric, or semiparametric. One suitable example of a statistical model which may be used to predict sequence-property characteristics is a linear regression model.

U.S. Provisional Patent Application Nos. 63/297,679, 63/320,067, 63/338,398, 63/338,433, and 63/339,450 describe exemplary models that are amenable to the methods described herein (e.g., the affinity and/or enrichment score data produced by the methods described herein) and are incorporated by reference herein.

### EXAMPLES

The following examples are merely illustrative and are not meant to limit any aspects of the present disclosure.

### Example 1: a Quantitative Affinity ACE (“qaACE”) Assay, as a Method for Sampling the Affinity Antibody Variants

Traditional antibody screening approaches explore only a small sequence space, which may confer suboptimal properties such as insufficient binding affinity, developability limitations, and poor immunogenicity profiles. In contrast, deep mutagenesis coupled with screening or selection allows for the exploration of a larger antibody sequence space, thereby potentially yielding more and better drug leads. However, deep mutagenesis comes with its own challenges. For example, most mutations degrade the binding affinity of antibodies rather than improve it, which greatly reduces screening efficiency. Moreover, the combinatorics of the antibody sequence variant space grows exponentially with mutational load (i.e. the number of mutations simultaneously introduced into each sequence variant) and quickly exceeds the capacity of experimental assays by orders of magnitude. Finally, with most antibody screening approaches, antibody sequence variant libraries can be screened for only one property at a time, which makes it difficult to simultaneously optimize for multiple properties. Simultaneous rather than sequential optimization of antibody properties is desirable because improving one property at a time may lead to degradation of a different property, a pitfall that can be avoided by taking all properties of interest into account concurrently. Deep learning methods have been proposed as a tool for overcoming the limitations of experimental screening capacity. The general approach involves training a model on a small amount of experimental binding data and using this to predict which sequences are most likely to improve binding. Several promising approaches have been proposed (See, e.g., Khan et al, arXiv: 2201.12570 [q-bio.BM] (2021); Jin et al, arXiv preprint arXiv: 2110.04624 (2021); Jin et al, Proceedings of the 39th International Conference on Machine Learning, PMLR 162:10217-10227 (2022); Luo et al, BioRxiv doi: 10.1101/2022.07.10.499510, (2022); Mahajan et al, BioRxiv doi: 10.1101/2022.06.06.494991, (2022); Jeffrey et al, Patterns, 3:100406 (2022); Shuai et al, BioRxiv doi: 10.1101/2021.12.13.472419 (2021), but only a few have had in-silico predictions validated in the lab (See, e.g., Mason et al, Nat Biomed Eng. 600-612 (2021); Saka et al, Sci Rep. 11 (1): 5852. (2021)). While sufficient as a proof of principle, such demonstrations are limited for practical design by the shortcomings of screening platforms used to generate training data: binary (rather than continuous) readouts with limited throughput. Overall, this limits the quantitative accuracy of the models and the ability to extrapolate to higher mutational loads. Here fully quantitative, high-throughput experimental binding affinity data was generated using a Quantitative Affinity Activity-specific Cell-Enrichment (qaACE) assay. In this Example, the qaACE assay is a Fluorescent-Activated Cell Sorting (FACS) method paired with deep sequencing that generates a quantitative affinity score for each screened variant. Variants are expressed intracellularly in native soluble form in SoluPro™ E. coli B Strain. The qaACE assay was applied to two different antibody-antigen pairs generating high-throughput data sets to train antibody-specific language models, such as the models described in U.S. Provisional Patent Application Nos. 63/297,679, 63/320,067, 63/338,398, 63/338,433, and 63/339,450.

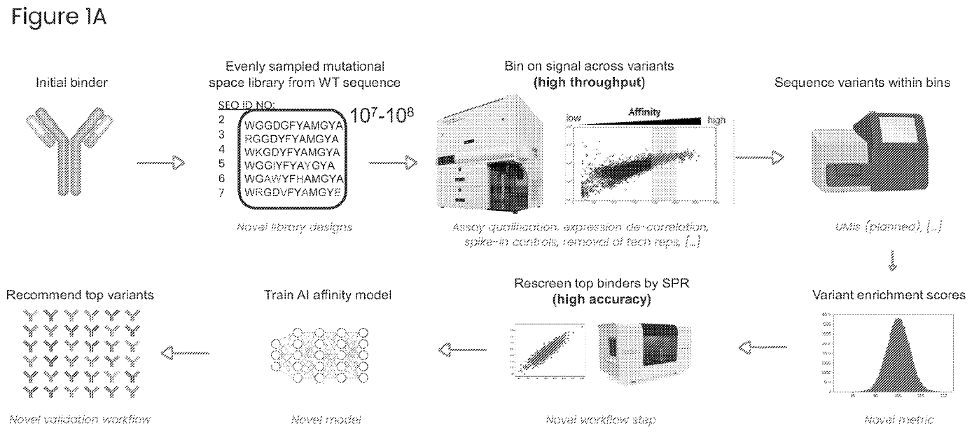

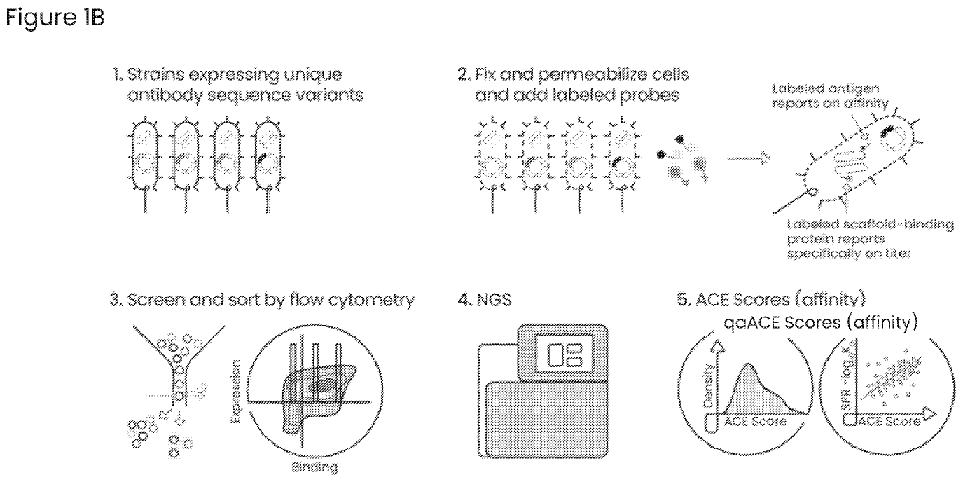

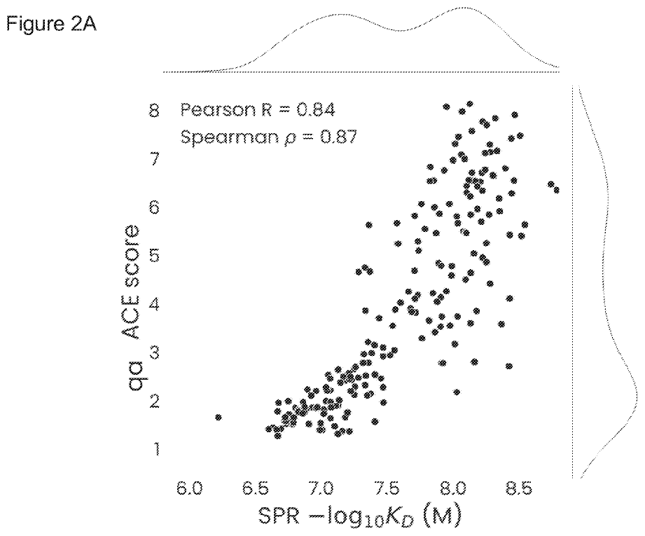

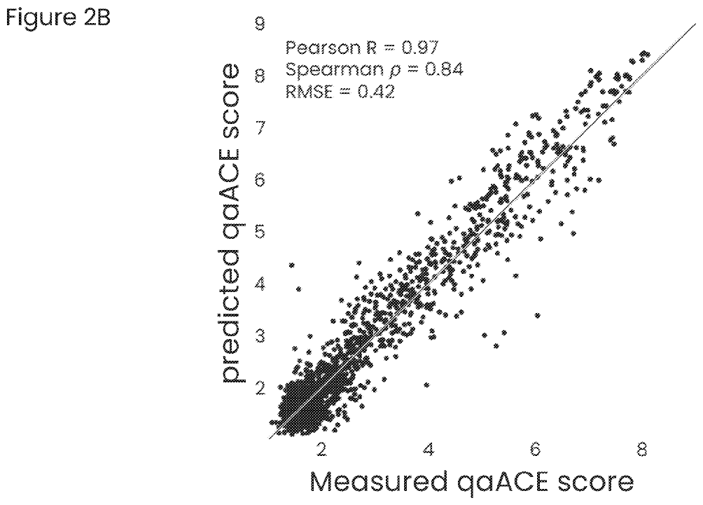

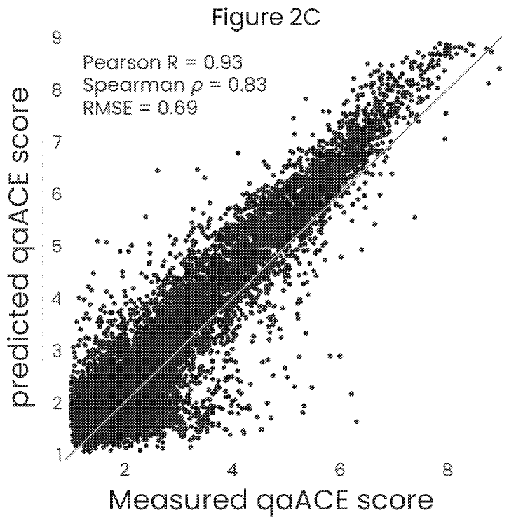

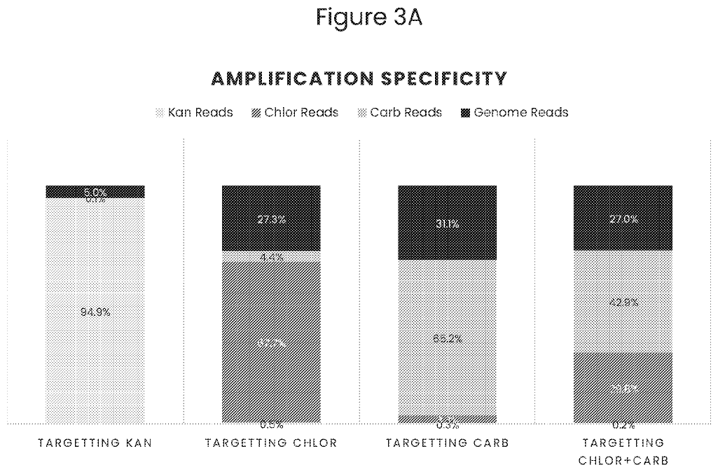

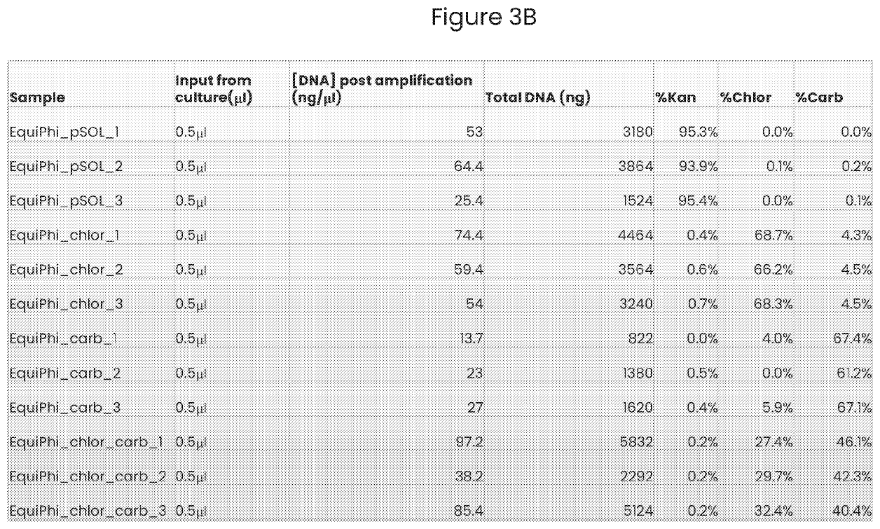

To generate high-throughput measurements of antibody variant binding affinity the qaACE assay was developed. FIG. 1 provides a general qaACE workflow. Cells expressing antibody variants were fixed, permeabilized, and stained with fluorescently labeled antigen and scaffold probes. These probes discriminate between the affinity and titer of the variants expressed within individual cells. The stained cell library was sorted and binned based on expression and affinity signals. The resulting sorted material was sequenced via Next-Generation Sequencing (NGS) and affinity scores were calculated based on read counts. qaACE affinity scores correlated strongly with SPR K_{D} measurements (FIG. 2A). The qaACE assay thus provides numerous advantages over existing methods for large scale antibody variant interrogation such as Tite-Seq (Adams et al, eLife, 5: e23156, (2016)), SORTCERY (Reich et al, J Mol Biol. 427(11): 2135-50 (2015)) and Phage Display (Chan et al, Int Immunol. 26 (12): 649-57 (2014)). First, qaACE utilizes SoluPro™ E. coli B Strain to solubly express antibodies intracellularly, avoiding binding artifacts associated with surface display format. Additionally, qaACE leverages genetic tools available for E. coli, enabling faster library generation cycles and increased transformation efficiency compared to other model organisms. Finally, the qaACE assay is a true screening method where all variants are measured regardless of affinity strength, as opposed to selections, such as phage display, where only high affinity binders are preferentially isolated.

### Materials and Methods

### Libraries of Antibody Variants

### Library Design

The heavy chain of trastuzamab was used:

HER2-targeting trastuzumab
(SEQ ID NO: 1)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS (Bostrom et al, Science.
323: 1610-1614 (2009))

Up to 3 simultaneous amino acid substitutions were introduced randomly in a parent antibody, in up to two CDRs, allowing all natural amino acids except cysteine. Cysteine residues were excluded from our library designs to avoid potential antibody structure liabilities. Mutagenesis of CDRH2 and CDRH3 was prioritized as these regions accommodate the highest density of paratope residues (Akbar et al, Cell Rep. 34 (11): 108856 (2021)). This mutagenesis strategy results in a combinatorial sequence space on the order of 10⁶-10⁷ variants.

### DNA Synthesis

DNA variants spanning CDRH2 and CDRH3 in a single oligonucleotide were synthesized using ssDNA oligos (IDT) or oligo pools (Twist). For Twist oligos, codons were randomly selected from the two most common in E. coli B strain (Nakamura et al, Nucleic Acids Res. 28 (1): 292 (2000)) for each variant, in which case two synonymous DNA sequences were synthesized (5 or 10 for parent antibody, calibrators, negative controls) for each amino acid variant. For IDT oligos, codon usage was identical for all variants, except at mutated positions where NNK degenerate codons were used.

TABLE 1
Antibody variant libraries.

| | Library | | trast-1 | trast-2 | trast-3 |
|---|---|---|---|---|---|
| Parent Antibody* | | | (T) | (T) | (T) |
| Screening | | | qaACE | SPR | qaACE |
| Design** | | | (A) | (B) | (E) |
| Mutated CDRH2 | | | — | — | 10 |
| Mutated CDRH3 | | | 8 | 8 | 10 |
| | | 1 | 142 | 23 | 315 |
| | Number | 2 | 8,789 | 191 | 4,054 |
| | of Mutations | 3 | — | — | 44,704 |
| | in Variants | 4 | — | — | 1,992 |
| | | 5 | — | — | 1,530 |

| | | | |
|---|---|---|---|
| No. of Measured Variants | 8,931 | 214 | 52,595 |

*Parent antibodies: (T)
**Design methods: (A)
Exhaustive sampling of combinatorial space single and double mutants in CDR3, (B) Near-uniform by affinity from trast-001, (C) Defined -log₁₀K_{D} by model predictions, (D) Uniform logK_{D} distribution by model predictions, (E) Random sampling of combinatorial space, (F) Defined -log₁₀K_{D} and naturalness by model predictions.

### Cloning

Library antibody variants were cloned and expressed in Fab format. Pools of degenerate oligonucleotides spanning framework region two, CDRH2, framework region three, CDRH3, and framework region four were ordered from Integrated DNA Technologies (IDT). Oligonucleotide pools were designed such that only CDRH2 and CDRH3 were subjected to NNK mutagenesis, while framework regions were held constant (parental sequence). Oligonucleotide pools were further designed such that CDRH2 and CDRH3 were each subjected to between one and three amino acid substitutions using NNK codons, with two and three amino acid substitution oligonucleotide pools encompassing all possible amino acid position combinations within a given CDR. Assembly PCR was carried out to recapitulate the region described above. Assembly reactions consisted of 0.04 UM of oligonucleotide pool material for each internal fragment, 4 UM of oligonucleotide pool material for each terminal fragment, and 1× Platinum SuperFi II Mastermix (ThermoFisher). Reactions were initially denatured at 98 °C for 30 s, followed by 20 cycles of 98 °C for 30 s; 60.5 °C for 30 s; 72 °C for 30 s; with a final extension of 72 °C for 10 min. PCR bands of the correct size were subsequently purified from a 1.25% agarose gel (Zymo Research Gel DNA Recovery Kit).

Amplification of Twist Bioscience's ssDNA oligo pools was carried out by PCR according to manufacturer recommendations with the exception that Platinum SuperFi II DNA polymerase (ThermoFisher) was used in place of KAPA polymerase. Briefly, 20 μl reactions consisted of 1× Platinum SuperFi II Mastermix, 0.3 μM each of forward and reverse primers, and 10 ng oligo pool. Reactions were initially denatured for 3 min at 95 °C followed by 13 cycles of: 95 °C for 20s; 66 °C for 20s; 72 °C for 15s; and a final extension of 72 °C for 1 min. DNA amplification was confirmed by agarose gel electrophoresis, and amplified DNA was subsequently purified (Zymo Research DNA Clean and Concentrate Kit).

To generate linearized trastuzumab Fab format vectors, PCR was carried out to split Absci's respective plasmid vectors into two fragments in a manner that provided cloning overlaps of approximately 30 nt on both the 5′ and 3′ ends with the amplified IDT (NNK) or Twist Biosciences libraries. Vector linearization reactions were digested with DPN1 (New England Biolabs) and purified from a 0.8% agarose gel (Zymo Research Gel DNA Recovery Kit) to eliminate parental vector carry through. Cloning reactions consisted of 50 fmol of each purified vector fragment, 100 fmol purified library (IDT or Twist Biosciences) insert, and 1× final concentration NEBuilder HiFi DNA Assembly (New England Biolabs). Reactions were incubated at 50 °C for two hours, and subsequently purified (Zymo Research DNA Clean and Concentrate Kit). Transformax Epi300 (Lucigen) E. coli were transformed by electroporation (BioRad MicroPulser) with the purified assembly reactions and grown overnight at 30 °C on LB agar plates containing 50 μg/ml kanamycin. The following morning colonies were scraped from LB plates and plasmids were extracted (Zymo Research Plasmid Midi Kit) and submitted for QC sequencing.

### QC

Antibody variant libraries for the ACE assay and SPR were amplified via PCR across the CDRH2 and CDRH3 region and sequenced via 2×150 nt Illumina NextSeq 1000 P2 platform with 20% PhiX. The PCR reaction used 10 nM primer concentration, Q5 2× master mix (NEB) and 1 ng of input DNA diluted in MGH20. Reactions were initially denatured at 98 °C for 3 min, followed by 30 cycles of 98 °C for 10 s; 59 °C for 30 s; 72 °C for 15 s; with a final extension of 72 °C for 2 min.

Sequencing reads were merged and analyzed as described in the qaACE primary analysis section below for distribution of mutations, variant representation, library complexity and recovery of expected sequences. Metrics included coefficient of variation of sequence representation, read share of top 1% most prevalent sequences and percentage of designed library sequences observed within the library.

Quantitative Affinity Activity-Specific Cell-Enrichment (qaACE) Assay
Antibody Expression in SoluPro™ E. coli B Strain

SoluPro™ E. coli B strain was transformed by electroporation (Bio-Rad MicroPulser) (See, e.g., WO/2014/025663 and WO/2017/106583). Cells were allowed to recover in 1 ml SOC medium for 90 minutes at 30 °C with 250 rpm shaking. Recovery outgrowths were centrifuged for 5 min at 8,000×g and the supernatant was removed. Resultant cell pellets were resuspended in 1 ml of induction media (IBM) supplemented with 50 μg/ml kanamycin and inducers and then added to 100 ml IBM containing 50 μg/ml kanamycin and inducers in a 1-L baffled flask. Antibody Fab induction was allowed to proceed at 30 °C with 250 rpm shaking for 24 h. At the end of 24 h, 1 ml aliquots of the induced culture were adjusted to 25% v/v glycerol and stored at −80 °C.

### Cell Preparation

High-throughput quantitative selection of antigen-specific Fab-expressing cells was adapted from the approach described in WO 2021/146626, which is incorporated herein by reference in its entirety. For staining, an OD600=2 of thawed glycerol stocks from induced cultures were transferred to 0.7 ml matrix tubes, centrifuged at 3300×g for 3 min, and resulting pelleted cells were washed three times with PBS+1 mM EDTA. Washed cells were thoroughly resuspended in 250 μl of 33 mM phosphate buffer (Na2HPO4) by pipetting then fixed by the further addition of 250 μl 32 mM phosphate buffer with 1.3% paraformaldehyde and 0.04% glutaraldehyde. After 40 min incubation on ice, cells were washed three times with PBS, resuspended in permeabilization buffer (20 mM Tris, 50 mM glucose, 10 mM EDTA, 5 μg/ml lysozyme) and permeabilized for 8 min on ice. Fixed and permeabilized cells were equilibrated by washing 3× in a stain buffer.

### Staining

Optimal permeabilization of SoluPro™ is sensitive to harvest conditions and the probe/fluorochrome of interest, so for each Fab reference strain and library pair, three different stain buffers were tested: 0.1% saponin buffer (1×PBS, 1 mM EDTA, 0.1% saponin, 1% heat-inactivated FBS), 0.5% triton buffer (1×PBS, 1 mM EDTA, 0.5% Triton X-100, 1% heat-inactivated FBS), and AlphaLISA immunoassay assay buffer (Perkin Elmer; 25 mM HEPES, 0.1% casein, 1 mg/ml dextran-500, 0.5% Triton X-100, and 0.05% kathon). Each probe was then titrated to determine the EC75 with the reference strain. Once buffer and probe conditions were established, fixed and permeabilized cells were resuspended in 250 μl stain buffer and transferred to a new matrix tube. A 2× concentration of the binding probe—50 nM human Her2: AF647 (Acro Biosystems) or 200 nM delta RBD with 6×HIS tag (Acro Biosystems, R&D Biosciences)—was prepared in stain buffer, then 250 μl probe was transferred to the prepared cells bringing the total stain volume to 500 μl. In some cases, an unlabeled competitor probe was included (IC30-80) to better resolve high affinity binders. Cells were incubated with the probe overnight (16 hrs) with end to end rotation at 4 °C protected from light. After incubation, cells were pelleted, washed 3× with PBS, and resuspended in 500 μl PBS containing 30 nM anti-kappa: AF488 (BioLegend, clone MHK-49) and, for HIS-tagged probes, 25 nM anti-HIS: AF647 (R&D Biosciences, clone AD1.1.10R). The expression and anti-HIS probes were incubated for 2 hrs as described above, then cells were washed 3× and resuspended in 500 μl PBS by thorough pipetting.

### Sorting

Libraries were sorted on FACSymphony S6 (BD Biosciences) instruments. Immediately prior to sorting, 50 μl prepped sample was transferred to a flow tube containing 1 ml PBS+3 μl propidium iodide. Aggregates, debris, and impermeable cells were removed with singlets, size, and PI₊ parent gating. To reduce expression bias, an additional parent gate was set on the mid 65% of peak expression positive cells. Collection gates were drawn to evenly sample the log range of binding signal with the far right gate set to collect enough events to collect ≥10,000 events over the allotted sort time, four to seven additional gates fractionating the positive binding signal, and one gate collecting the binding negative population. Libraries were sorted simultaneously on two instruments with photomultipliers adjusted to normalize fluorescence intensity, and the collected events processed independently as technical replicates.

### Next-Generation Sequencing

Cell material from various gates was collected in a diluted PBS mixture (VWR), in 1.5-ml tubes (Eppendorf). Post sort samples were spun down at 3,800 g and tube volume was normalized to 20 μl. Amplicons for sequencing were generated from the CDRH2 and CDRH3 region via a two-phase PCR, using collected cell material directly as a template. During the initial PCR phase, unique molecular identifiers (UMIs) and partial Illumina adapters were added to the CDRH2 and CDRH3 amplicon via 4 PCR cycles. The second phase PCR added the remaining portion of the Illumina sequencing adapter and the Illumina i5 and i7 sample indices. The initial PCR reaction used 1 nm UMI primer concentration, Q5 2× master mix (NEB) and 20 μl of sorted cell material input suspended in diluted PBS (VWR). Reactions were initially denatured at 98 °C for 3 min, followed by cycles of 98 °C for 10 s; 59 °C for 30 s; 72 °C for 30 s; with a final extension of 72 °C for 2 min. Following the initial PCR, 0.5 UM of the secondary sample index primers were added to each reaction tube. Reactions were then denatured at 98 °C for 3 min, followed by 29 cycles of 98 °C for 10 s; 62 °C for 30 s; 72 °C for 15 s; with a final extension of 72 °C for 2 min. After the 2nd PCR, samples were run on a 2% agarose gel at 75 V for 60 min and the proper length band was excised and purified using the Zymoclean Gel DNA Recovery Kit (Zymo Research). Resulting DNA samples were quantified by Qubit fluorometer (Invitrogen), normalized and pooled. Pool size was verified via Tapestation 1000 HS and was sequenced on an Illumina NextSeq 1000 P2 (2×150 nt) with 20% PhiX.

qaACE Analysis

### Preprocessing

In order to arrive at a quantitative binding score, the sequencing reads were passed through a series of computational processing and quality control steps. Paired-end reads were merged using FLASH2 (Magoč T, Salzberg S L. Bioinformatics. 27 (21): 2957-63 (2011)) with the maximum allowed overlap set according to the amplicon size and sequencing reads length (150 bases for all the libraries described in this manuscript). The downstream UMI tag (last 8 bases) was moved to the beginning of the read, and UMI Collapse tool (Liu D. PeerJ. 16; 7: e8275. (2019)) was used in FASTQ mode to remove any PCR duplicates. Fully identical sequences were only considered to be duplicates. The primers from both ends of the merged read were removed using Cutadapt tool (Martin, Marcel. EMBnet.journal: 17 (2011)), discarding reads where either primers were not detected. The reads across all of the FACS sorting gates were aggregated, and aligned to the reference sequence (wild-type version of the amplicon) in amino acid space. Alignment was done using the Needleman-Wunsch algorithm implemented in Biopython (Cock et al., Bioinformatics: 25 1422-1423 (2009)). PairwiseAligner, mode global, match_score 5, mismatch_score-4, open_gap_score-20, extend_gap_score-1; parameters were chosen by manual inspection across a number of processed libraries). The reads were then subjected to a set of quality assurance filters. (1) all reads where the mean base quality is below 20, or where any individual base in the region of interest has a quality score below 20, were dropped; (2) sequences (in DNA space) seen fewer than 10 times (i.e. in less than 10 unique molecules following UMI deduplication) across all of the gates were discarded. (1) Sequences that align to the reference with a low score (defined as less than 0.6 of the score obtained by aligning the reference to itself); (2) sequences containing stop codons outside of the region of interest and (3) sequences containing frame-shifting insertions or deletions, were all flagged. Flagged sequences were not included in any mutation-related statistics, but are used for count normalization when the binding score is calculated. The workflow also runs FastQC (Andrews, S. (2010).

https://bibsonomy.org/bibtex/f230a919c34360709aa298734d63dca3) and MultiQC (Ewels et al. Bioinformatics. 32:3047-3048 (2016)), to obtain the common sequencing quality control metrics. For the remaining sequences, the count within each gate (the number of times a sequence is seen in the gate) was normalized by dividing it with the total number of reads in the gate and multiplying by 1 million. Finally, a binding score (qaACE score or enrichment score) was assigned to each unique DNA sequence by taking a weighted average of the normalized counts across the sorting gates. For all the experiments in this manuscript, the weights were assigned linearly: the gate with the lowest signal getting the weight of 1, and the gate with the highest signal getting the weight equal to the total number of gates used in the experiment.

QC and Determination of gaACE Scores

Following the above described standardized processing workflow, each dataset was further restricted to the set of sequences in the respective library design. Finally, since each amino-acid sequence in the library received multiple measurements, from multiple synonymous DNA variants and multiple replicate FACS sorts, these measurements were aggregated into a single data-point by taking the mean value. This information was used as an additional quality control step (checking form consistency across the replicates), and to discard noisy data, by discarding sequences where the standard deviation across the measurements is above a manually derived threshold of 1.

### Surface Plasmon Resonance (SPR)

Antibody Expression in SoluPro™ E. coli B Strain

Individual SoluPro™ E. coli B strain colonies expressing antibody Fab variants were inoculated in LB media in 96-well deep blocks (Labcon) and grown at 30 °C for 24 hrs to create seed cultures for inducing expression. Seed cultures were then inoculated in IBM media (4.5 g/L Potassium Phosphate monobasic, 13.8 g/L Ammonium Sulfate, 20.5 g/L yeast extract, 20.5 g/L glycerol, 1.95 g/L Citric Acid) containing inducers and supplements (260 UM Arabinose, 50 μg/mL Kanamycin, 8 mM Magnesium Sulfate, 1 mM Propionate, 1× Korz trace metals) in 96-well deep block and additionally grown at 30 °C for 24 hrs. Post induction samples were transferred to 96-well plates (Greiner Bio-One), pelleted and lysed in 50 μL lysis buffer (1× BugBuster protein extraction reagent containing 0.01 KU Benzonase 499 Nuclease and 1× Protease inhibitor cocktail). Plates were incubated for 15-20 min at 30 °C then centrifuged to remove insoluble debris. After lysis samples were adjusted with 200 μL SPR running buffer (10 mM HEPES, 150 mM NaCl, 3 mM EDTA, 0.01% w/v Tween-20, 0.5 mg/mL BSA) to a final volume of 260 UL and filtered into 96-well plates. Lysed samples were then transferred from 96-well plates to 384-well plates for high-throughput SPR using a Hamilton STAR automated liquid handler. Colonies were prepared in two sets of independent replicates prior to lysis and each replicate was measured in two separate experimental runs. In some instances, single replicates were used, as indicated.

### SPR Experiments

High-throughput SPR experiments were conducted on a microfluidic Carterra LSA SPR instrument using SPR running buffer (10 mM HEPES, 150 mM NaCl, 3 mM EDTA, 0.01% w/v Tween-20, 0.5 mg/mL BSA) and SPR wash buffer (10 mM HEPES, 150 mM NaCl, 3 mM EDTA, 0.01% w/v Tween-20). Carterra LSA SAD200M chips were pre-functionalized with 20 μg/mL biotinylated antibody capture reagent for 10 mins prior to conducting experiments. Lysed samples in 384-well blocks were immobilized onto chip surfaces for 10 mins followed by a 1 min washout step for baseline stabilization. Antigen binding was conducted using the non-regeneration kinetics method with a 5 min association phase followed by a 15 min dissociation phase. For analyte injections, six leading blanks were introduced to create a consistent baseline prior to monitoring antigen binding kinetics. After the leading blanks, five concentrations of HER2 extracellular domain antigen (ACRO 518 Biosystems, prepared in three-fold serial dilution from a starting concentration of 500 nM), were injected into the instrument and the time series response was recorded. In most experiments, measurements on individual DNA variants were repeated four times. Typically each experiment run consisted of two complete measurement cycles (ligand immobilization, leading blank injections, analyte injections, chip regeneration) which provided two duplicate measurement attempts per clone per run. In most experiments, technical replicates measured in separate runs further doubled the number of measurement attempts per clone to four.

### Sensorgram Baseline Subtraction

Sensorgrams were generated from raw data using the Carterra Kinetics GUI software application provided with the Carterra LSA instrument. Sensorgram response values vs. time for 384 regions of interest (ROIs) on the Carterra chip were corrected using a double-referencing and alignment technique implemented by the Carterra manufacturer. This technique incorporates both the time-synchronous response of an interspot reference region adjacent to the ROI, as well as the non-synchronous response from a leading blank buffer injection flowing over the same ROI during an earlier experiment run cycle, to estimate and subtract a background response. Corrected sensorgrams were exported from the Kinetics software package for offline analysis.

### Kinetic Binding Parameters

Kinetic binding parameters were estimated via non-linear regression using a standard 1:1 binding model which was modified by the incorporation of a vector of to parameters each unique to one analyte concentration. For a single analyte concentration, the association phase model is:

$$\begin{matrix}c \underline{a} R \underline{max} \\ R ( t , {c}_{a} ) = \\ {c}_{a} + KD\end{matrix} [ 1 - {}_{e} - ( cakon + kof f ) ( t - tc ) ]$$

where
t=time
t_{c}=concentration-dependent time offset
   Cₐ=analyte concentration
kₒₙ=forward (association) reaction rate constant
k_{off}=backward (dissociation) reaction rate constant
K_{D}=k_{off}/kₒₙ
   Rₘₐₓ=asymptotic maximum instrument response.

The additional concentration-dependent time offset parameter to was needed because of the unique measurement system that Carterra uses, in which successive association phase measurements at each new analyte concentration are attempted before the analyte from the previous phase has fully dissociated, leading to response curves which do not begin from zero response at t=0. The time offset parameters represent the projected time intercept of each association response curve; i.e., the amount of time prior to the start of the association phase, at which the measurement would have had to begin in order to reach the actual observed response at t=0. The dissociation phase was modeled as a standard decaying exponential curve:

R(t,Cₐ)=Rde-kof f(t−td−tc)

where
td=start time of dissociation phase measurement
   Rd=final estimated response value R (td, Cₐ) from association equation.

The regression was conducted using R-language scripts (R Core Team. https://R-project.org). Minpack.Im (Ezhov et al., https://cran.r-project.org/web/packages/minpack.Im/minpack.Im.pdf), an R-ported copy of MINPACK-1 (Moré, J. J In: Watson, G. A., (ed.) Numerical Analysis (Lee. Notes Math. 630:105-116. (1977); Moré et al., https://osti.gov/biblio/5171554), a FORTRAN-based software package which implements the Levenberg-Marquardt (Levenberg K., Qu. Appl. Math. 2:164-168 (1944); Marquardt, D. J. Soc. Indust. Appl. Math. 11:2 (1963)) non-linear least squares parameter search algorithm, was used to conduct the parameter search.

### QC

SPR fits were excluded if any of the following criteria was satisfied:
less than 3 analyte concentrations providing usable fits
handling errors as noted by operator
non-physical fits (such as an upward-sloping dissociation-phase signal, even after sensorgram baseline subtraction)
non-convergent fits
a signal-to-noise ratio less than 10
a tc value, for the highest analyte concentration ca included in the fit (typically 500 nM), such that tc<−300 s or to >0 s
failed NGS
non-clonal sequence (dominant sequence less than 100 times as abundant as secondary sequence when the Levenshtein distance between the two is greater than 2)
sequence does not match any designed variant in the synthesized library (within a sequence identity tolerance to accommodate sequencing errors)
KD and K_{off} were-log 10 transformed, while kₒₙ was log 10 transformed. For all three kinetic parameters, plot labels refer to the log 10-transformation without specifying whether the sign was positive or negative. Distributions of kinetic parameters were visually inspected for absence of significant batch effects.

Multiple measurements of the same antibody variant (usually (a) duplicate serial measurements of the same clone in the same SPR run; (b) technical replicates of the same clone from duplicate 384-well plates measured in separate runs; (c) two DNA variants with identical translation, when available; and (d) independent clones of a variant) were averaged. Variants whose −log 10KD measurements showed a coefficient of variation greater than 5% upon aggregation were dropped.

### Next-Generation Sequencing

To identify the DNA sequence of individual antibody variants evaluated in SPR, NGS was carried out on measured variants. Individual colonies were picked from LB agar plates containing 50 g/ml Kanamycin (Teknova) into 96 deep well plates containing 1 ml LB media (Teknova). The culture plates were grown overnight in a 30 °C shaker incubator. 200 μl of overnight culture was transferred into new 96 well plates (Labcon) and spun down at 3500 g. A portion of the pelleted material was transferred into 96 well PCR (Thermo-Fisher) plate via pinner (Fisher Scientific) which contained reagents for performing an initial phase PCR of a two-phase PCR for addition of Illumina adapters and sequencing. Reaction volumes used were 25 μl. During the initial PCR phase, unique molecular identifiers (UMIs) and partial Illumina adapters were added to CDRH2 and CDRH3 amplicons via 4 PCR cycles. The second phase PCR added the remaining portion of the Illumina sequencing adapter and the Illumina i5 and i7 sample indices. The initial PCR reaction used 0.45UM UMI primer concentration, 12.5 μl Q5 2× master mix (NEB). Reactions were initially denatured at 98 °C for 3 min, followed by 4 cycles of 98 °C for 10 s; 59 °C for 30 s; 72 °C for 30 s; with a final extension of 72 °C for 2 min. Following the initial PCR, 0.5 UM of the secondary sample index primers were added to each reaction tube. Reactions were then denatured at 98 °C for 3 min, followed by 29 cycles of 98 °C for 10 s; 62 °C for 30 s; 72 °C for 15 s; with a final extension of 72 °C for 2 min. Reactions were then pooled into a 1.5 ml tube (Eppendorf). Pooled samples were size selected with a 1×AMPure XP (Beckman Coulter) bead procedure. Resulting DNA samples were quantified by Qubit fluorometer. Pool size was verified via Tapestation 1000 HS and was sequenced on an Illumina MiSeq Micro (2×150 nt) with 20% PhiX.

After sequencing, amplicon reads were merged corresponding to their sample indices. Merging was performed by custom Python scripts. Instances of unique amplicon sequences within each sample were counted. Next, custom R scripts were applied to calculate sequence frequency and distance metric thresholds for quality filtering. CDR region sequences were then extracted from the amplicon sequences. These CDR sequences were then combined with companion Carterra SPR measurements.

### Example 2: Selective Rolling Circle Amplification

The present Example demonstrates that selective rolling circle amplification (sRCA) can be used to amplify a plasmid of interest in its entirety from very low input quantities of cells. Unique suites of primers were designed to selectively amplify any plasmid backbone, regardless of the insert's identity. For each backbone type, candidate primers were created in the conserved regions of antibiotic resistance markers and/or the flanking regions. The candidate primers were tested against other plasmid backbones to remove any candidates that would bind to more than one backbone type. Primers were also screened against the E. coli genome to reduce off-target amplification of genomic DNA. Therefore, only primers that exclusively bind their target backbone were selected. The targeted nature of the primer design allowed the amplification of single plasmid types from cells containing multiple plasmid types, while avoiding off-target amplification of other plasmids or genomic DNA. The 70,000 base pair strand displacement capabilities of EquiPhi29 DNA polymerase allows for primer binding in a relatively small portion of the plasmid and still effectively amplifies the entire sequence.

sRCA has advantages over both (1) the standard paradigm of extraction to transformation to regrowth methodology, as well as (2) non-specific rolling circle amplification that uses random hexamer primers.

For the standard paradigm, sRCA reduces the time required to process and generate data from ACE sorts (i.e., cells sorted following an ACE assay or qaACE assay as described herein) by up to 50%. Direct amplification of DNA prevents any bias introduced by transformation and regrowth in K-strain E. coli. Chaperone libraries may be especially susceptible to skewing under such conditions. The selective nature of the primer suites allows for exclusive amplification of the plasmid of interest without interference by other plasmids in the system or genomic DNA. The system may be limited by the salt-tolerance of the enzyme. For example, the FACS sorting machines used in a typical ACE assay use 1×PBS as a buffer and, when concentrated, can inhibit amplification reactions. The careful preprocessing laid out in subsection (C) of this example effectively reduced the salt concentration in the system to a level acceptable for sufficient amplification of template DNA.

For non-specific amplification, initial testing of RCA used random hexamer primers. While significant amplification was achieved, sequence data analysis revealed that up to 80% of sequencing reads were from unwanted amplification of the E. coli genome. Such a large amount of non-target reads required adjustment to allocate more reads per sample, which reduced the net gain created by amplification. Such large amounts of genomic DNA also make the amplified product unusable for long-read sequencing on a PacBio Sequel platform. This platform is limited in throughput, making each individual read very valuable. A comparison of random hexamer amplification to sRCA showed drastic improvements in % target reads using sRCA. With sRCA and fragment size selection via PippenHT, >90% on-target reads by PacBio are achievable compared to ~70-80% with miniprepped DNA and 50-80% with random hexamer amplification.

The following four experiments demonstrate that selective rolling circle amplification (SRCA) can be used to amplify a plasmid of interest in its entirety from very low input quantities of cells.

### A. Accuracy and Specificity

Overnight culture, amplification, and sequencing were conducted to verify the accuracy and specificity of the selective primers for sRCA. A plasmid backbone was tested, which can contain any of three antibiotic resistance markers: PL2945 (Kan), PL3133 (Chlor), and/or PL3137 (Carb). As shown in FIG. 2, amplification of plasmids expressing the Kan resistance marker was highly specific when compared to the other antibiotic resistance markers. 94.9% of the reads from the amplification were specific for the plasmid encoding the Kan resistance marker, with only 5% genomic reads and almost no off-target enrichment. In contrast, the Chlor and Carb resistance marker plasmids yielded 27.3% and 31.1% genomic reads, respectively, and off-target reads were also present in both samples. While the Kan resistant plasmid and corresponding primers were superior in terms of amplification specificity, the Chlor- and Carb-specific reads were still present at a sufficient fraction to resolve the plasmid sequence. Thus, sRCA is an optimal amplification platform for all the specific plasmid backbones listed herein with a Kan resistance marker. Furthermore, optimization of primers for Carb- and Chlor-resistant plasmid backbones is possible if expansion of the suite of antibiotic resistance markers is desired.

### B. Genomic Contamination

Standard minipreps typically result in 5-25% of reads mapping to the E. coli genome, rather than the plasmid of interest. Alternative sample preparation methods can be employed to reduce these off-target reads. These methods were investigated by preparing plasmid samples with a miniprep protocol, either alone or with samples treated with Plasmid-Safe, and sRCA-amplified plasmid, either alone or in conjunction with PippenHT size selection. Plasmid-Safe™ ATP-dependent DNase is an exonuclease that degrades linear (genomic) DNA while leaving circular (plasmid) DNA unaffected. PippenHT Size selection is a process by which a device runs sample DNA through gel electrophoresis alongside a ladder of known size. The operator indicates what size ranges are to be collected and the machine diverts sample DNA of the indicated size to a collection well by monitoring the lane containing the ladder that has known run times associated with its known sizes. Sample DNA that is not within the specified size range is collected in a waste well.

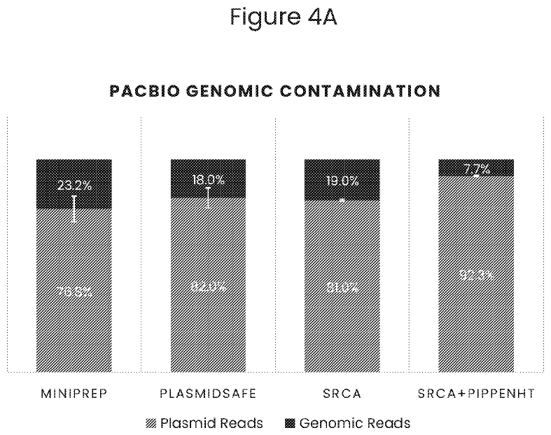

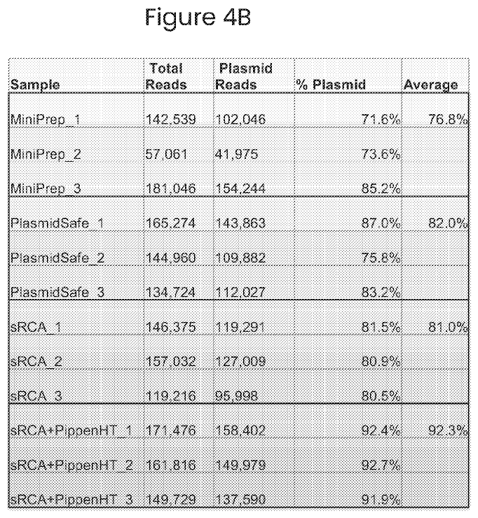

Both pretreatment of miniprep samples with Plasmid-Safe™ and PippenHT size selection of sRCA samples increased the fraction of the product that consisted of plasmid-specific reads. As shown in FIG. 4A, Miniprepped samples alone had 76.8% plasmid-specific reads, but treatment with Plasmid-Safe™ increased that fraction to 82.0% plasmid-specific reads. Addition of Plasmid-Safe™ raised the fraction of plasmid-specific reads obtained via miniprep to be roughly equivalent with the fraction of plasmid-specific reads for SRCA alone, which was 81.0%. However, implementation of PippenHT size selection prior to sRCA increased the average fraction of plasmid-specific reads to 92.3%, making these conditions optimal for plasmid-specific amplification while reducing the fraction of genomic reads to 7.7% of the total reads recorded.

### C. Post ACE-Sort Amplification

The current standard of practice for sequencing the products of an ACE (e.g., ACE assay or qaACE assay as described herein) sort is time-consuming, laborious, and subject to bottlenecks and bias due to the transformation and regrowth steps in E. coli K strain cells prior to sequencing. In the standard of practice protocol, after an ACE sort is completed and a population is selected the cells must be digested, the plasmid extracted, bacteria must be transformed with the plasmid and grown, and the resultant bacterial population must be miniprepped. This standard of practice may be improved with an alternative workflow described herein. The alternative workflow can be faster and requires less active processing than the standard of practice, although this alternative workflow may present some risk of amplification bias.

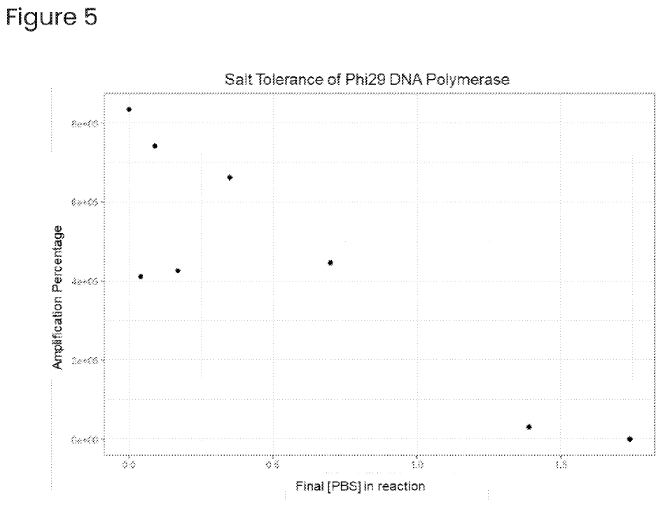

To reduce amplification bias, the alternative workflow may incorporate pre-processing steps prior to the sRCA reaction. In one embodiment, these steps can involve transferring the samples to a PCR plate, adding additional water, centrifugation of the plate and removal of roughly 70% of the supernatant, further reducing the volume of liquid via evaporation, and inputting the resultant product into the sRCA reaction. This alternative methodology can overcome the problem of salt inhibition of sRCA enzymes. PBS may be used as sheath fluid for the qaACE assay. However, PBS contains concentrations of salts that may at least partially inhibit the amplification reaction. The alternative workflow's pre-processing steps described herein serve to dilute and remove as much salt as possible allowing sufficient amplification of the plasmid, as shown in FIG. 5. With the alternative workflow properly employed, the post-ACE sort amplification may be conducted in a single day. Overnight amplification reactions yield far more amplified DNA than is necessary for sequencing applications.

D. Integration of sRCA Sequence Data with qaACE

As described herein, the present Example and workflow allows association (e.g., connection of DNA/Amino Acid sequence with the scores generated by the ACE and Carterra workflows. Post-sort sequencing from an ACE assay allows for the calculation of scores for each DNA/AA variant. Sequencing done on clones that undergo the SPR Carter workflow connect DNA/AA variant with a measured K_{D}. Thus, the data can be merged where a DNA/AA sequence can be assigned both ACE and K_{D} measurement scores by merging two separate data frames by the DNA/AA sequence identified independently in each workflow) of DNA and/or amino acid sequences with ACE data (qaACE) and data from Carterra. In one iteration of the experiment, the amino acid sequence derived from sRCA may be associated with a number of analyte readouts, including but not limited to: kₐ, k_{d}, K_{D}, Rₘₐₓ, and Res SD.

### Example 3: Flow Cytometry Gating and Correlation of qaACE Scores to Measured Affinity Constants

Continuing the methods as described in Example 1 above, the present example describes a flow cytometry sorting workflow and correlation of qaACE scores to measured affinity constants.

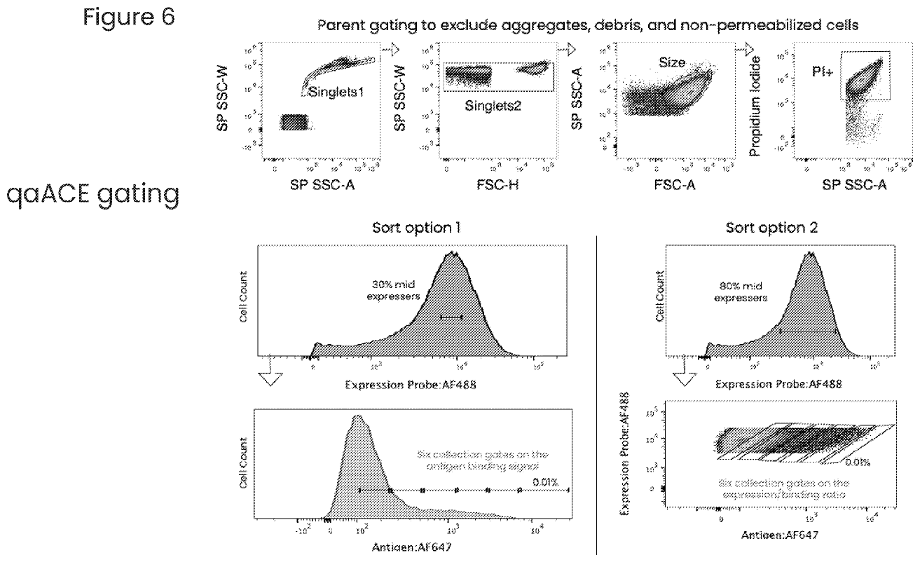

Fixed, permeabilized, and stained cells are sorted by the expressed lead protein's (fab) affinity for antigen. The flow cytometry gating scheme is shown in FIG. 6. After parent gating to reduce aggregates, debris, and non-permeabilized cells, bias to antigen binding signal from expression variability is controlled through an additional parent gate on the 30% mid expressers. Six collection gates were then used to bin evenly across the log range of the antigen signal (sort option 1). Alternatively, cells may be collected on the ratio of the expression signal over binding signal (sort option 2).

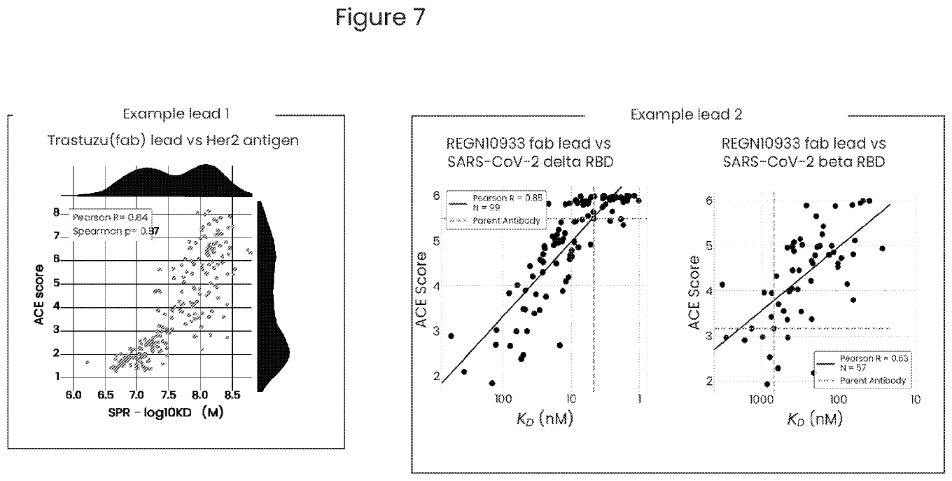

After sorting, unique molecular identifiers are added to flank the CDR region. Collected material was then amplified and sequenced. Read counts weighted by distribution in the sort gates were used to assign ACE scores to each variant. ACE scores show a strong correlation to SPR-measured affinity constants (KDs), permitting the high throughput ACE data (≥50 k sequences) to be used for model training. FIG. 7 shows the ACE score vs SPR KD comparison for libraries from two parental molecules, trastuzumab fab (left) against Her2 antigen and REGN10933 fab (right) against a high affinity SARS-COV-2 antigen (delta variant) and low affinity SARS-COV-2 antigen (beta variant).

What is claimed is:

1. A method for generating training data for a machine learning model comprising:
f) expressing a biomolecule variant library in host cells;
g) measuring: (i) expression levels and (ii) affinity values to a binding partner of interest of two or more biomolecule variants expressed in (b);
h) sorting the host cells into a distribution of cell subpopulations based on the measured expression levels and measured affinity values; thereby collecting cells across an affinity distribution;
i) sequencing the biomolecule variants expressed from the collected cells of (c);
j) calculating an enrichment score for each sequenced biomolecule variant, wherein said enrichment score and said biomolecule variant sequence is capable of training a machine learning model capable of performing sequence-based affinity predictions.

2. The method of claim 1, wherein the library of biomolecule variants is generated by randomly mutating a nucleic acid encoding a reference biomolecule.

3. The method of claim 1 or 2, wherein the library of biomolecule variants is generated by random mutagenesis, error-prone PCR mutagenesis, oligonucleotide-directed mutagenesis, cassette mutagenesis, shuffling, saturation mutagenesis, homology-directed mutagenesis, Activation Induced Cytidine Deaminase (AID) mediated mutagenesis, or transposon mutagenesis.

4. The method of any one of claims 1-3, wherein the library of biomolecule variants comprises at least 10⁴-10⁷ unique biomolecule variant sequences.

5. The method of any one of claims 1-4, wherein the library of biomolecule variants are displayed on the host cell surface.

6. The method of any one of claims 1-5, wherein the library of biomolecule variants are expressed and retained in the host cell cytoplasm.

7. The method of claim any one of claims 1-6, wherein the host cells are Escherichia coli cells.

8. The method of claim 7, wherein the Escherichia coli cells are Escherichia coli 521 cells.

9. The method of claim 6 or 7 wherein the Escherichia coli cells comprises one or more or all of:
i) an alteration of gene function of at least one gene encoding a transporter protein for an inducer of at least one inducible promoter;
j) a reduced level of gene function of at least one gene encoding a protein that metabolizes an inducer of at least one inducible promoter;
k) a reduced level of gene function of at least one gene encoding a protein involved in biosynthesis of an inducer of at least one inducible promoter;
l) an altered gene function of a gene that affects the reduction/oxidation environment of the host cell cytoplasm;
m) a reduced level of gene function of a gene that encodes a reductase;
n) at least one expression construct encoding at least one disulfide bond isomerase protein;
o) at least one polynucleotide encoding a form of DsbC lacking a signal peptide; and/or
p) at least one polynucleotide encoding Ervlp.

10. The method of claim 1, wherein step (c) optionally additionally measures one or more of binding specificity, biological activity, stability, and/or solubility of the expressed biomolecule variants.

11. The method of any one of claims 1-9, wherein affinity is quantified by measuring binding dissociation constant (K_{D}) of a biomolecule variant to the binding partner of interest.

12. The method of claim 10 wherein the binding partner of interest is a fluorescently labeled antigen.

13. The method of any one of claims 1-12, wherein expression level of the biomolecule variants is quantified by measuring anti-IgG-binding capacity.

14. The method of any one of claims 1-12, wherein expression level of the biomolecule variants is quantified using an anti-IgG antibody conjugated to a fluorophore.

15. The method of any one of claims 1-12, wherein expression level of the biomolecule variants is quantified by measuring a non-antigen binding capacity.

16. The method of any one of claims 1-15, wherein the measuring in step (c) and sorting in step (d) comprises a fluorescence-activated cell sorting (FACS) assay.

17. The method of any one of claims 1-16, optionally further comprising measuring binding affinity of the sequenced biomolecule variants prior to calculating an enrichment score.

18. The method of claim 17, wherein the binding affinity is measured using an assay selected from the group consisting of a Surface Plasmon Resonance (SPR) based binding assay, Biolayer Interferometry and/or flow cytometry derived binding curves.

19. The method of any one of claims 1-18, wherein the sequencing of step (e) is obtained by a method selected from the group consisting of deep sequencing, next generation sequencing, Long read nanopore sequencing, Single Molecule Real-Time long read sequencing (pacbio).

20. The method of any one of claims 1-19, wherein the sequencing of step (e) is obtained by a method selected from the group consisting of deep sequencing, next generation sequencing, Long read nanopore sequencing, Single Molecule Real-Time long read sequencing (Pacbio).

21. The method of any one of claims 17-20, wherein nucleic acids encoding the biomolecule variants are modified prior to sequencing to comprise barcode sequences comprising unique molecular identifiers (UMIs).

22. The method of claim 1, wherein the biomolecule variants are selected from a group consisting of a monoclonal antibody, a bispecific antibody, a multispecific antibody, a humanized antibody, a chimeric antibody, a camelised antibody, a single domain antibody, a single-chain Fvs (ScFv), a single chain antibody, a Fab fragment, a F(ab′) fragment, a disulfide-linked Fvs (sdFv), or an anti-idiotypic (anti-Id) antibody.

23. The method of claim 1, wherein the biomolecule variants are selected from a group consisting of a monoclonal antibody, a bispecific antibody, a multispecific antibody, a humanized antibody, a chimeric antibody, a camelised antibody, a single domain antibody, a single-chain Fvs (ScFv), a single chain antibody, a Fab fragment, a F(ab′) fragment, a disulfide-linked Fvs (sdFv), or an anti-idiotypic (anti-Id) antibody.

24. The method of claim 1, wherein the biomolecule variants are selected from a group consisting of a peptide, a polypeptide, a protease, an oxidoreductase, a transferase, a hydrolase, a lyase, an isomerase, a ligase, an enzyme, an antibody, a cytokine, a chemokine, a nucleic acid, a metabolite, a small molecule (<1 kDa) and a synthetic molecule.

25. A method for generating training data for a machine learning model comprising:
a) expressing a biomolecule variant library in host cells;
b) measuring: (i) expression levels and (ii) affinity values to a binding partner of interest of two or more biomolecule variants expressed in (b);
c) sorting the host cells into a distribution of cell subpopulations based on the measured expression levels and measured affinity values; thereby collecting cells across an affinity distribution;
d) isolating nucleic acids encoding the biomolecule variants from the collected host cells of (c), amplifying said nucleic acids using selective rolling circle amplification (SRCA), and sequencing nucleic acids encoding the biomolecule variants; and
e) calculating an enrichment score for each sequenced biomolecule variant, wherein said enrichment score and said biomolecule variant sequence is capable of training a machine learning model capable of performing sequence-based affinity predictions.


SEQUENCE LISTING (7 sequences)

SEQ ID NO: 1 - protein, 120 aa, synthetic construct [sequence omitted]

SEQ ID NO: 2 - protein, 12 aa, synthetic construct
WGGDGFYAMGYA

SEQ ID NO: 3 - protein, 12 aa, synthetic construct
RGGDYFYAMGYA

SEQ ID NO: 4 - protein, 12 aa, synthetic construct
WKGDYFYAMGYA

SEQ ID NO: 5 - protein, 12 aa, synthetic construct
WGGIYFYAYGYA

SEQ ID NO: 6 - protein, 12 aa, synthetic construct
WGAWYFHAMGYA

SEQ ID NO: 7 - protein, 12 aa, synthetic construct
WRGDVFYAMGYE